(19) United States
(12) Patent Application Publication
(10) Pub. No.: US 2018/0119164 A1
(43) Pub. Date: May 3, 2018

(54) Nucleic Acid Molecule and Uses Thereof

(71) Applicant: Universität für Bodenkultur Wien, Vienna (AT)

(72) Inventors: Andreas Loos, Vienna (AT); Hertha Steinkellner, Vienna (AT); Lukas Mach, Langenzersdorf (AT)

(21) Appl. No.: 15/568,113

(22) PCT Filed: Apr. 20, 2016
(86) PCT No.: PCT/EP2016/058743

§ 371 Date: Oct. 20, 2017

(30) Foreign Application Priority Data
Apr. 20, 2015 (EP) 15164196.6

(51) Int. Cl. C12N 15/82; C12N 9/10

(52) U.S. Cl. CPC C12N 15/8258; C12N 9/13; C12N 15/8243; C12N 15/8257; C12Y 208/0202

(57) ABSTRACT

The present invention relates to a nucleic acid molecule encoding
a) a modified tyrosylprotein sulfotransferase of a wildtype tyrosylprotein sulfotransferase, wherein the cytoplasmic transmembrane stem (CTS) region of the wild-type tyrosylprotein sulfotransferase is replaced by a heterologous CTS region, or
b) a fusion protein comprising a catalytically active fragment of a tyrosylprotein sulfotransferase fused to a heterologous CTS region.

The present invention relates to means and methods for producing sulfated polypeptides in plants.

Most proteins, in particular in eukaryotic systems, undergo posttranslational modifications. Such modifications are important because they may alter physical and chemical properties, conformation, distribution, stability, activity, folding and consequently, function of the proteins. Moreover, many proteins show (significant) activity only if they are posttranslationally modified. Higher eukaryotes perform a variety of posttranslational modifications like methylation, sulfation, phosphorylation, lipid addition and glycosylation. Secreted proteins, membrane proteins and proteins targeted to vesicles or certain intracellular organelles are likely to be glycosylated. The most common glycosylation is N-linked glycosylation where oligosaccharides are added to asparagine residues found in AsnX-Ser/Thr sequences of proteins. Another type of glycosylation is O-linked glycosylation which involves simple oligosaccharide chains or glycosaminoglycan chains. Sulfation is known to play an important role in strengthening protein-protein interactions. Types of human proteins which often undergo sulfation include adhesion molecules, G-protein-coupled receptors, coagulation factors, serine protease inhibitors, extracellular matrix proteins and hormones. The target amino acid of the sulfation reaction is tyrosine and the reaction is, thus, called tyrosine sulfation. In the course of the sulfation reaction a sulfate group is added to a tyrosine residue of a target protein molecule. Sulfation is catalyzed by tyrosylprotein sulfotransferase (TPST) in the Golgi apparatus of eukaryotic cells.

WO 2014/093702 discloses sulfated HIV-1 envelope proteins. These proteins can be used to treat or prevent HIV-1 infections.

Cimbro R et al. (PNAS 111(2014):3152-3157) discovered that tyrosine sulfation in a variable loop region of the HIV-1 gp120 protein is responsible for the protein stability and for modulating the neutralization sensitivity.

Rosenberg Y et al. (PLOS ONE 10(2015):e0120451-1) studied the influence of mutations on the immunogenicity of broadly neutralizing HIV monoclonal antibodies in macaques.

Strasser R et al. (Curr Opin Biotechn 30(2014):95-100) is a review article about the glycosylation of plant-produced recombinant proteins.

Loos A et al. (Front Plant Sci 5(2014):523-1) is a review article about glycosylation of proteins in plants. Therein, targeting mechanisms of glycosyltransferases are discussed.

Moore K. L. (PNAS 106(14741-14742) discusses protein tyrosine sulfation in plants and animals.

Grabenhorst E et al. (J Biol Chem 274(1999):36107-36116) found that the cytoplasmic, transmembrane and stem regions of glycosyltransferases specify their in vivo sublocalisation and stability in the Golgi.

Pejchal R et al. (PNAS 107(2010):11483-11488) discovered that monoclonal antibody PG16 comprises a subdomain which mediates neutralization of HIV-1.

As mentioned above posttranslational modifications influence or are often essential for the biological activity of polypeptides and proteins. This has to be considered when host cells are selected to be used for the recombinant production of polypeptides and proteins. Especially the recombinant expression of animal polypeptides and proteins in plant or yeast cells often requires a modification of the host cell to adapt the posttranslational modification to the polypeptides and proteins to be expressed. Plant cells, for instance, have already been modified to recombinantly produce proteins having an animal like glycosylation. However, strategies to sulfate recombinantly produced proteins of animal origin in plant cells, for instance, have not been established yet.

It is therefore an object of the present invention to provide methods and means enabling a host cell, preferably of nonanimal origin, to sulfate a recombinant protein preferably derived from an animal.

The present invention relates to a nucleic acid molecule encoding
a) a modified tyrosylprotein sulfotransferase of a wild-type tyrosylprotein sulfotransferase, wherein the cytoplasmic transmembrane stem (CTS) region of the wild-type tyrosylprotein sulfotransferase is replaced by a heterologous CTS region, or
b) a fusion protein comprising a catalytically active fragment of a tyrosylprotein sulfotransferase fused to a heterologous CTS region.

It turned out that the replacement of a naturally occurring CTS region of a tyrosylprotein sulfotransferase by a CTS region of another polypeptide or protein results in an increase of the protein sulfation rate in a host cell expressing such a modified tyrosylprotein sulfotransferase. Furthermore, the recombinant expression of the modified tyrosylprotein sulfotransferase of the present invention enables host cells to sulfate recombinant proteins and polypeptides although the corresponding wild-type host cells do not have such sulfation activities.

“Modified tyrosylprotein sulfotransferase”, as used herein, refers to a tyrosylprotein sulfotransferase which has a different amino acid sequence as naturally occurring tyrosylprotein sulfotransferases (“wild-type tyrosylprotein sulfotransferases”). These modified tyrosylprotein sulfotransferases comprise instead of their naturally occurring CTS region a heterologous CTS region.

As used herein, a “cytoplasmic transmembrane stem (CTS) region” and a “CTS region” or a “cytoplasmic transmembrane stem (CTS) domain” and a “CTS domain” comprises the cytoplasmic tail, transmembrane domain and stem region of Golgi-resided proteins and polypeptides. CTS regions mediate sorting of the proteins and polypeptides attached thereto into the different functional compartments of the Golgi apparatus.

CTS regions of Golgi-resided proteins can be identified using methods well-known in the art, such as, for example, hydropathy plot analysis and sequence alignments with known CTS regions. A CTS region may consist of a substantial part of a CTS region, such as at least 50% or at least 60% or at least 70% or at least 80% or at least 90% of a CTS region. The CTS region/domain may consist of 1 to 100, preferably 5 to 90, more preferably 10 to 80, more preferably 15 to 70, more preferably 15 to 60, more preferably 20 to 50, more preferably 25 to 45, more preferably 30 to 40, amino acid residues located at the Cor N-terminus of a Golgi-resided protein or polypeptide.

The term “replaced by”, as used herein, means that the cytoplasmic transmembrane stem (CTS) region of the wild-type tyrosylprotein sulfotransferase is at least partially, preferably entirely, exchanged by a heterologous CTS region, whereby “heterologous” means that the CTS region is not naturally occurring in said wild-type tyrosylprotein sulfotransferase.

“Fusion protein” as used herein refers to a fusion of two or more amino acid sequences that are not naturally linked together. The fusion of the amino acid sequences can either be made by chemically linking ex vivo synthesized amino acid sequences. In an embodiment, the amino acid sequences are an in-frame translational fusion, i.e. correspond to a recombinant molecule expressed from a nucleic acid sequence in a prokaryotic or eukaryotic expression system. The term “fusion protein” as used herein refers also to a protein which may be created through genetic engineering from two or more proteins/peptides coding sequences joined together in a single coding sequence. In general, this is achieved by creating a “fusion gene”, a nucleic acid that encodes and expresses the fusion protein. For example, a fusion gene that encodes a fusion protein may be made by removing the stop codon from a first DNA sequence encoding the first protein, then appending a DNA sequence encoding the second protein in frame. The resulting fusion gene sequence will then be expressed by a cell as a single fusion protein. Fusion proteins may include a linker (or “spacer”) sequence which can promote appropriate folding and activity of each domain of the fusion protein.

“Catalytically active fragment” or “enzymatically active fragment”, as used herein, refers to a polypeptide fragment that contains the catalytically active domain of a tyrosylprotein sulfotransferase sufficient to exhibit activity. A catalytically active fragment is the portion of a tyrosylprotein sulfotransferase that, under appropriate conditions, can exhibit catalytic activity and is able to transfer a sulfate residue to a tyrosyl residue of a peptide, polypeptide or protein. Typically, a catalytically active fragment is a contiguous sequence of amino acid residues of a tyrosylprotein sulfotransferase that contains the catalytic domain and required portions for recognizing the substrate to be sulfated. A preferred enzymatically/catalytically active fragment of a tyrosylprotein sulfotransferase lacks amino acid residues 1 to 100, preferably 5 to 90, more preferably 10 to 80, more preferably 15 to 70, more preferably 15 to 60, more preferably 20 to 50, more preferably 25 to 45, more preferably 30 to 40, even more preferably 1 to 39, of a wild-type tyrosylprotein sulfotransferase (e.g. SEQ ID No. 1).

“Nucleic acid”, as used herein, refers to a deoxyribonucleotide (DNA) or a ribonucleotide polymer (RNA) in either singleor double-stranded form. The nucleic acid molecule of the present invention is preferably a DNA molecule.

The modified tyrosylprotein sulfotransferase and the fusion protein according to the present invention comprise a heterologous CTS region, i.e. a CTS region which is not naturally occurring in the corresponding wild-type tyrosylprotein sulfotransferase. This heterologous CTS region is preferably a CTS region of a protein or polypeptide of a plant or animal.

According to a preferred embodiment of the present invention the plant CTS region is a CTS region of a protein or polypeptide of Arabidopsis thaliana, Nicotiana spp, Physcomitrella patens or medicago truncatula.

In a further embodiment of the present invention animal CTS region is a mammalian CTS region, preferably of a protein or polypeptide of a rat, more preferably of a protein or polypeptide of Rattus norvegicus, or of human origin.

The heterologous CTS region is preferably a CTS region of a glycosyltransferase or a tyrosylprotein sulfotransferase.

According to a preferred embodiment of the present invention the glycosyltransferase is selected from the group consisting of a fucosyltransferase, preferably an α1,3-fucosyltransferase, more preferably an α1,3-Fucosyltransferase 11, and a sialytransferase, preferably an α2,6-sialytransferase.

According to a further preferred embodiment of the present invention the tyrosylprotein sulfotransferase is a plant or animal tyrosylprotein sulfotransferase.

According to another preferred embodiment of the present invention the animal tyrosylprotein sulfotransferase is a mammalian, preferably human or mouse, tyrosylprotein sulfotransferase, a nematode, preferably a Caenorhabditis elegans, tyrosylprotein sulfotransferase or an insect, preferably a Drosophila melanogaster, tyrosylprotein sulfotransferase.

The plant tyrosylprotein sulfotransferase is preferably a Arabidopsis thaliana tyrosylprotein sulfotransferase.

The heterologous CTS region can be fused either N- or C-terminally to a catalytically active fragment of a tyrosylprotein sulfotransferase.

According to a preferred embodiment of the present invention the wild-type tyrosylprotein sulfotransferase comprises or consists of an amino acid sequence selected from the group consisting of SEQ ID No. 1, SEQ ID No. 13, SEQ ID No. 15, SEQ ID No. 16, SEQ ID No. 19, SEQ ID No. 20, SEQ ID No. 23 and SEQ ID No. 25.

According to a further preferred embodiment of the present invention the wild-type tyrosylprotein sulfotransferase is encoded by a nucleic acid sequence selected from the group consisting of SEQ ID No. 2, SEQ ID No. 14, SEQ ID No. 17, SEQ ID No. 18, SEQ ID No. 21, SEQ ID No. 22, SEQ ID No. 24 and SEQ ID No. 26.

Particularly preferred is a human wild-type tyrosylprotein sulfotransferase comprising amino acid sequence SEQ ID No. 1 or 13, whereby amino acid residues 1 to 50, preferably 1 to 45, more preferably 1 to 42, more preferably 1 to 41, more preferably 1 to 40, more preferably 1 to 38, more preferably 1 to 37, more preferably 1 to 35, more preferably 1 to 30, in particular 1 to 39, of SEQ ID No. 1 and amino acid residues 1 to 50, preferably 1 to 45, more preferably 1 to 42, more preferably 1 to 41, more preferably 1 to 39, more preferably 1 to 38, more preferably 1 to 35, more preferably 1 to 30, in particular 1 to 40, of SEQ ID No. 13 represent the CTS region:

(SEQ ID No. 1)
MVGKLKQNLLLACLVISSVTVFYLGQHAMECHHRIEERSQPVKLESTRTT
VRTGLDLKANKTFAYHKDMPLIFIGGVPRSGTTLMRAMLDAHPDIRCGEE
TRVIPRILALKQMWSRSSKEKIRLDEAGVTDEVLDSAMQAFLLEIIVKHG
EPAPYLCNKDPFALKSLTYLSRLFPNAKFLLMVRDGRASVHSMISRKVTI
AGFDLNSYRDCLTKWNRAIETMYNQCMEVGYKKCMLVHYEQLVLHPERWM
RTLLKFLQIPWNHSVLHHEEMIGKAGGVSLSKVERSTDQVIKPVNVGALS
KWVGKIPPDVLQDMAVIAPMLAKLGYDPYANPPNYGKPDPKIIENTRRVY
KGEFQLPDFLKEKPQTEQVE
(SEQ ID No. 13)
MRLSVRRVLLAAGCALVLVLAVQLGQQVLECRAVLAGLRSPRGAMRPEQE
ELVMVGTNHVEYRYGKAMPLIFVGGVPRSGTTLMRAMLDAHPEVRCGEET
RIIPRVLAMRQAWSKSGREKLRLDEAGVTDEVLDAAMQAFILEVIAKHGE
PARVLCNKDPFTLKSSVYLSRLFPNSKFLLMVRDGRASVHSMITRKVTIA
GFDLSSYRDCLTKWNKAIEVMYAQCMEVGKEKCLPVYYEQLVLHPRRSLK
LILDFLGIAWSDAVLHHEDLIGKPGGVSLSKIERSTDQVIKPVNLEALSK
WTGHIPGDVVRDMAQIAPMLAQLGYDPYANPPNYGNPDPFVINNTQRVLK
GDYKTPANLKGYFQVNQNSTSSHLGSS

A nucleic acid sequence encoding the human wild-type tyrosylprotein sulfotransferase may comprise SEQ ID No. 2 or 14, whereby nucleotide residues 1 to 150, preferably 1 to 135, more preferably 1 to 126, more preferably 1 to 123, more preferably 1 to 120, more preferably 1 to 114, more preferably 1 to 111, more preferably 1 to 105, more preferably 1 to 90, in particular 1 to 117, of SEQ ID No. 2 and nucleotide residues 1 to 150, preferably 1 to 135, more preferably 1 to 126, more preferably 1 to 123, more preferably 1 to 117, more preferably 1 to 114, more preferably 1 to 105, more preferably 1 to 90, in particular 1 to 120, of SEQ ID No. 14 encode the CTS region:

(SEQ ID No. 2)
atggttggaaagctgaagcagaacttactattggcatgtctggtgattag
ttctgtgactgtgttttacctgggccagcatgccatggaatgccatcacc
ggatagaggaacgtagccagccagtcaaattggagagcacaaggaccact
gtgagaactggcctggacctcaaagccaacaaaacctttgcctatcacaa
agatatgcctttaatatttattggaggtgtgcctcggagtggaaccacac
tcatgagggccatgctggacgcacatcctgacattcgctgtggagaggaa
accagggtcattccccgaatcctggccctgaagcagatgtggtcacggtc
aagtaaagagaagatccgcctggatgaggctggtgttactgatgaagtgc
tggattctgccatgcaagccttcttactagaaattatcgttaagcatggg
gagccagccccttatttatgtaataaagatccttttgccctgaaatcttt
aacttacctttctaggttattccccaatgccaaatttctcctgatggtcc
gagatggccgggcatcagtacattcaatgatttctcgaaaagttactata
gctggatttgatctgaacagctatagggactgtttgacaaagtggaatcg
tgctatagagaccatgtataaccagtgtatggaggttggttataaaaagt
gcatgttggttcactatgaacaacttgtcttacatcctgaacggtggatg
agaacactcttaaagttcctccagattccatggaaccactcagtattgca
ccatgaagagatgattgggaaagctgggggagtgtctctgtcaaaagtgg
agagatctacagaccaagtaatcaagccagtcaatgtaggagctctatca
aaatgggttgggaagataccgccagatgttttacaagacatggcagtgat
tgctcctatgcttgccaagcttggatatgacccatatgccaacccaccta
actacggaaaacctgatcccaaaattattgaaaacactcgaagggtctat
aagggagaattccaactacctgactttcttaaagaaaaaccacagactga
gcaagtggagtag
(SEQ ID No. 14)
atgcgcctgtcggtgcggagggtgctgctggcagccggctgcgccctggt
cctggtgctggcggttcagctgggacagcaggtgctagagtgccgggcgg
tgctggcgggcctgcggagcccccggggggccatgcggcctgagcaggag
gagctggtgatggtgggcaccaaccacgtggaataccgctatggcaaggc
catgccgctcatcttcgtgggtggcgtgcctcgcagtggcaccacgttga
tgcgcgccatgctggacgcccaccccgaggtgcgctgcggcgaggagacc
cgcatcatcccgcgcgtgctggccatgcgccaggcctggtccaagtctgg
ccgtgagaagctgcggctggatgaggcgggggtgacggatgaggtgctgg
acgccgccatgcaggccttcatcctggaggtgattgccaagcacggagag
ccggcccgcgtgctctgcaacaaggacccatttacgctcaagtcctcggt
ctacctgtcgcgcctgttccccaactccaagttcctgctgatggtgcggg
acggccgggcctccgtgcactccatgatcacgcgcaaagtcaccattgcg
ggctttgacctcagcagctaccgtgactgcctcaccaagtggaacaaggc
catcgaggtgatgtacgcccagtgcatggaggtaggcaaggagaagtgct
tgcctgtgtactacgagcagctggtgctgcaccccaggcgctcactcaag
ctcatcctcgacttcctcggcatcgcctggagcgacgctgtcctccacca
tgaagacctcattggcaagcccggtggtgtctccctgtccaagatcgagc
ggtccacggaccaggtcatcaagcctgttaacctggaagcgctctccaag
tggactggccacatccctggggatgtggtgcgggacatggcccagatcgc
ccccatgctggctcagctcggctatgacccttatgcaaacccccccaact
atggcaaccctgaccccttcgtcatcaacaacacacagcgggtcttgaaa
ggggactataaaacaccagccaatctgaaaggatattttcaggtgaacca
gaacagcacctcctcccacttaggaagctcgtgatttccagatctccgca
aatgacttcattgccaagaagagaagaaaatgcatttaa

Particularly preferred is a mammalian tyrosylprotein sulfotransferase of a mouse comprising amino acid sequence SEQ ID No. 15 or SEQ ID No. 16, whereby amino acid residues 1 to 50, preferably 1 to 45, more preferably 1 to 42, more preferably 1 to 41, more preferably 1 to 40, more preferably 1 to 38, more preferably 1 to 37, more preferably 1 to 35, more preferably 1 to 30, in particular 1 to 39, of SEQ ID No. 15 and amino acid residues 1 to 65, preferably 1 to 60, more preferably 1 to 55, more preferably 1 to 54, more preferably 1 to 52, more preferably 1 to 50, more preferably 1 to 45, more preferably 1 to 40, in particular 1 to 53, of SEQ ID No. 16 represent the CTS region:

(SEQ ID No. 15)
MVGKLKQNLLLACLVISSVTVFYLGQHAMECHHRIEERSQPARLENPKAT
VRAGLDIKANKTFTYHKDMPLIFIGGVPRSGTTLMRAMLDAHPDIRCGEE
TRVIPRILALKQMWSRSSKEKIRLDEAGVTDEVLDSAMQAFLLEVIVKHG
EPAPYLCNKDPFALKSLTYLARLFPNAKFLLMVRDGRASVHSMISRKVTI
AGFDLNSYRDCLTKWNRAIETMYNQCMEVGYKKCMLVHYEQLVLHPERWM
RTLLKFLHIPWNHSVLHHEEMIGKAGGVSLSKVERSTDQVIKPVNVGALS
KWVGKIPPDVLQDMAVIAPMLAKLGYDPYANPPNYGKPDPKILENTRRVY
KGEFQLPDFLKEKPQTEQVE
(SEQ ID No. 16)
MRRAPWLGLRPWLGMRLSVRKVLLAAGCALALVLAVQLGQQVLECRAVLG
GTRNPRRMRPEQEELVMLGADHVEYRYGKAMPLIFVGGVPRSGTTLMRAM
LDAHPEVRCGEETRIIPRVLAMRQAWTKSGREKLRLDEAGVTDEVLDAAM
QAFILEVIAKHGEPARVLCNKDPFTLKSSVYLARLFPNSKFLLMVRDGRA
SVHSMITRKVTIAGFDLSSYRDCLTKWNKAIEVMYAQCMEVGRDKCLPVY
YEQLVLHPRRSLKRILDFLGIAWSDTVLHHEDLIGKPGGVSLSKIERSTD
QVIKPVNLEALSKWTGHIPRDVVRDMAQIAPMLARLGYDPYANPPNYGNP
DPIVINNTHRVLKGDYKTPANLKGYFQVNQNSTSPHLGSS

A nucleic acid sequence encoding the mammalian, preferably mouse, tyrosylprotein sulfotransferase may comprise SEQ ID No. 17 or SEQ ID No. 18, whereby nucleotide residues 1 to 150, preferably 1 to 135, more preferably 1 to 126, more preferably 1 to 123, more preferably 1 to 120, more preferably 1 to 114, more preferably 1 to 111, more preferably 1 to 105, more preferably 1 to 90, in particular 1 to 117, of SEQ ID No. 17 and nucleotide residues 1 to 195, preferably 1 to 180, more preferably 1 to 165, more preferably 1 to 162, more preferably 1 to 156, more preferably 1 to 150, more preferably 1 to 135, more preferably 1 to 120, in particular 1 to 159, of SEQ ID No. 18 encode the CTS region:

(SEQ ID No. 17)
atggttgggaagctgaagcagaacttactcttggcgtgtctggtgattag
ttctgtgaccgtgttttacctgggccagcatgccatggagtgccatcacc
gaatagaggaacgtagccagccagcccgactggagaaccccaaggcgact
gtgcgagctggcctcgacatcaaagccaacaaaacattcacctatcacaa
agatatgcctttaatattcatcgggggtgtgcctcggagcggcaccacac
tcatgagggctatgctggacgcacatcctgacatccgctgtggagaggaa
accagggtcatccctcgaatcctggccctgaagcagatgtggtcccggtc
cagtaaagagaagatccgcttggatgaggcgggtgtcacagatgaagtgc
tagattctgccatgcaagccttccttctggaggtcattgttaaacatggg
gagccggcaccttatttatgtaacaaagatccgtttgccctgaaatcctt
gacttaccttgctaggttatttcccaatgccaaatttctcctgatggtcc
gagatggccgggcgtcagtacattcaatgatttctcggaaagttactata
gctggctttgacctgaacagctaccgggactgtctgaccaagtggaaccg
ggccatagaaaccatgtacaaccagtgtatggaagttggttataagaaat
gcatgttggttcactatgaacagctcgtcttacaccctgaacggtggatg
agaacgctcttaaagttcctccatattccatggaaccattccgttttgca
ccatgaagaaatgatcgggaaagctgggggagtttctctgtcaaaggtgg
aaagatcaacagaccaagtcatcaaacccgtcaacgtgggggcgctatcg
aagtgggttgggaagatacccccggacgtcttacaagacatggccgtgat
tgcacccatgctcgccaagcttggatatgacccatacgccaatcctccta
actacggaaaacctgaccccaagatccttgaaaacaccaggagggtctat
aaaggagaatttcagctccctgactttctgaaagaaaaaccccagacgga
gcaagtggagtaa
(SEQ ID No. 18)
atgaggcgggccccctggctgggcctgcgaccctggctgggcatgcgcct
gtcggtgcgtaaggtgctgctggccgccggctgtgctctggccctggtgc
tcgctgtgcagcttgggcagcaagtactggagtgccgggcggtgctcggg
ggcacacggaacccacggaggatgcggccggagcaggaggaactggtgat
gctcggcgccgaccacgtggagtaccgctatggcaaggccatgccactca
tctttgtgggcggcgtgccacgcagtggcaccacgctcatgcgcgccatg
ttggacgcacacccagaggtgcgctgtggggaggagacgcgcatcatccc
tcgtgtgctggccatgcggcaggcctggaccaagtctggccgtgagaagc
tgcggctggacgaggcaggtgtgacggatgaggtgctggacgcggccatg
caggccttcattctggaggtgatcgccaagcacggcgaaccagcccgcgt
gctgtgtaacaaggaccccttcacactcaagtcatccgtctacctggcac
gcctgttccccaactccaaattcctgctaatggtgcgtgacggccgggcg
tccgtgcactccatgatcacgcgcaaggtcaccatcgcgggctttgacct
cagcagctaccgagactgcctcaccaagtggaacaaggccatcgaggtga
tgtacgcacagtgcatggaggtgggcagggacaagtgcctgcccgtgtac
tatgagcagttggtgctgcacccccggcgctcactcaaacgcatcctgga
cttcctgggcatcgcctggagtgacacagtcctgcaccacgaggacctca
ttggcaagcctgggggcgtctccttgtccaagatcgagcggtccacggac
caggtcatcaaaccggtgaacttggaagctctctccaagtggacgggcca
catccctagagacgtggtgagggatatggcccagattgcccccatgctgg
cccggcttggctatgacccgtatgcgaatccacccaactatgggaacccc
gaccccattgtcatcaacaacacacaccgggtcttgaaaggagactataa
aacgccagccaatctgaaaggatattttcaggtgaaccagaacagcacct
ccccacacctaggaagttcgtga

Particularly preferred is a nematode tyrosylprotein sulfotransferase of Caenorhabditis elegans comprising amino acid sequence SEQ ID No. 19 or SEQ ID No. 20, whereby amino acid residues 1 to 50, preferably 1 to 45, more preferably 1 to 42, more preferably 1 to 41, more preferably 1 to 40, more preferably 1 to 38, more preferably 1 to 37, more preferably 1 to 35, more preferably 1 to 30, in particular 1 to 39, of SEQ ID No. 19 and amino acid residues 1 to 50, preferably 1 to 45, more preferably 1 to 42, more preferably 1 to 40, more preferably 1 to 38, more preferably 1 to 35, more preferably 1 to 30, of SEQ ID No. 20 represent the CTS region:

(SEQ ID No. 19)
MRKNRELLLVLFLVVFILFYFITARTADDPYYSNHREKFNGAAADDGDES
LPFHQLTSVRSDDGYNRTSPFIFIGGVPRSGTTLMRAMLDAHPEVRCGEE
TRVIPRILNLRSQWKKSEKEWNRLQQAGVTGEVINNAISSFIMEIMVGHG
DRAPRLCNKDPFTMKSAVYLKELFPNAKYLLMIRDGRATVNSIISRKVTI
TGFDLNDFRQCMTKWNAAIQIMVDQCESVGEKNCLKVYYEQLVLHPEAQM
RRITEFLDIPWDDKVLHHEQLIGKDISLSNVERSSDQVVKPVNLDALIKW
VGTIPEDVVADMDSVAPMLRRLGYDPNANPPNYGKPDELVAKKTEDVHKN
GAEWYKKAVQVVNDPGRVDKPIVDNEVSKL
(SEQ ID No. 20)
MRAILDAHPDVRCGGETMLLPSFLTWQAGWRNDWVNNSGITQEVFDDAVS
AFITEIVAKHSELAPRLCNKDPYTALWLPTIRRLYPNAKFILMIRDARAV
VHSMIERKVPVAGYNTSDEISMFVQWNQELRKMTFQCNNAPGQCIKVYYE
RLIQKPAEEILRITNFLDLPFSQQMLRHQDLIGDEVDLNDQEFSASQVKN
SINTKALTSWFDCFSEETLRKLDDVAPFLGILGYDTSISKPDYSTFADDD
FYQFKNFYS

A nucleic acid sequence encoding the nematode, preferably Caenorhabditis elegans, tyrosylprotein sulfotransferase may comprise SEQ ID No. 21 or SEQ ID No. 22, whereby nucleotide residues 1 to 150, preferably 1 to 135, more preferably 1 to 126, more preferably 1 to 123, more preferably 1 to 120, more preferably 1 to 114, more preferably 1 to 111, more preferably 1 to 105, more preferably 1 to 90, in particular 1 to 117, of SEQ ID No. 21 and nucleotide residues 1 to 150, preferably 1 to 135, more preferably 1 to 126, more preferably 1 to 120, more preferably 1 to 114, more preferably 1 to 105, more preferably 1 to 90, of SEQ ID No. 22 encode the CTS region:

(SEQ ID No. 21)
atgagaaaaaatcgagagttgctactcgtcctcttcctcgtcgtttttat
actattctattttattactgcgagaactgcagacgacccgtactacagta
accatcgggagaaattcaatggtgccgccgccgacgacggcgacgagtcg
ttaccttttcatcaattaacgtcagtacgaagtgatgatggatacaatag
aacgtctcctttcatattcataggtggtgttcctcgctccggtacaactc
tgatgcgtgcgatgcttgacgctcatccagaagtcagatgtggtgaggag
acacgtgtcattccacgcatcctgaatctacggtcacaatggaaaaagtc
ggaaaaggagtggaatcgactgcagcaggctggagtgacgggtgaagtga
ttaacaatgcgatcagctcgtttatcatggagataatggttggccacgga
gatcgggctcctcgtctctgcaacaaggatccattcacaatgaaatcagc
cgtctacctaaaagaactcttcccaaatgccaaatatcttctaatgatcc
gtgatggacgggccaccgtgaatagtataatctcacgaaaagtcacaatt
accggattcgatttgaacgatttccgtcaatgcatgacgaaatggaatgc
ggcaattcaaataatggtagatcagtgtgaatcggttggagagaaaaatt
gtttgaaagtgtattatgagcagctggtgctacatccggaagcacaaatg
cggcgaattacagagtttttggatattccgtgggatgataaagtgctgca
ccatgagcagcttattggaaaagatatttctttatcgaatgtggaacgga
gctcggatcaagtcgttaaaccggttaatcttgatgctcttatcaaatgg
gttggaacgattcctgaggatgttgttgctgatatggattcggttgcgcc
gatgttaaggagattaggatatgatccgaatgcaaatccaccaaactatg
gaaaacccgacgaactagtcgcgaaaaaaacggaagatgttcataaaaat
ggagccgaatggtacaagaaagcagttcaagtggtcaacgatcccggccg
cgtcgataaaccaattgttgataatgaagtatcgaaatta
tag
(SEQ ID No. 22)
atgagagctattctagatgcacatccggatgttcgatgtggcggtgaaac
catgctgcttccaagtttccttacatggcaagcaggctggcggaatgatt
gggtcaataattcaggaattactcaggaagtatttgacgacgctgtttca
gcattcatcactgagatagtcgcgaagcacagtgaactagcacctcgtct
gtgcaacaaggatccatacaccgcattgtggcttccgactattcgccgac
tgtacccgaatgcaaagtttattctgatgattcgagatgctcgtgccgta
gttcattcaatgatagaaagaaaagtaccagttgctgggtataatacgtc
tgatgaaatttcaatgtttgttcagtggaatcaggagcttcgaaaaatga
cttttcaatgcaataatgcgccagggcaatgcataaaagtatattatgaa
cgactgattcaaaaacctgcggaagaaatcctacgtatcaccaacttcct
ggatctgccattttcccagcaaatgctaagacatcaagatttaattggag
acgaagttgatttaaacgatcaagaattctctgcatcacaagttaaaaac
tcgataaacactaaagccttaacctcgtggtttgattgttttagtgaaga
aactctacgaaaacttgatgacgtggcaccttttttgggaattcttggat
acgatacgtcgatttcaaaacccgattattccacatttgcggatgacgat
ttttaccaatttaaaaatttttattcttaa

Particularly preferred is an insect tyrosylprotein sulfotransferase of Drosophila melanogaster comprising amino acid sequence SEQ ID No. 23, whereby amino acid residues 1 to 60, preferably 1 to 55, more preferably 1 to 50, more preferably 1 to 49, more preferably 1 to 48, more preferably 1 to 46, more preferably 1 to 45, more preferably 1 to 40, more preferably 1 to 35, in particular 1 to 47, of SEQ ID No. 23 represent the CTS region:

(SEQ ID No. 23)
MRLPYRNKKVTLWVLFGIIVITMFLFKFTELRPTCLFKVDAANELSSQMV
RVEKYLTDDNQRVYSYNREMPLIFIGGVPRSGTTLMRAMLDAHPDVRCGQ
ETRVIPRILQLRSHWLKSEKESLRLQEAGITKEVMNSAIAQFCLEIIAKH
GEPAPRLCNKDPLTLKMGSYVIELFPNAKFLFMVRDGRATVHSIISRKVT
ITGFDLSSYRQCMQKWNHAIEVMHEQCRDIGKDRCMMVYYEQLVLHPEEW
MRKILKFLDVPWNDAVLHHEEFINKPNGVPLSKVERSSDQVIKPVNLEAM
SKWVGQIPGDVVRDMADIAPMLSVLGYDPYANPPDYGKPDAWVQDNTSKL
KANRMLWESKAKQVLQMSSSEDDNTNTIINNSNNKDNNNNQYTINKIIPE
QHSRQRQHVQQQHLQQQQQQHLQQQQHQRQQQQQQREEESESEREAEPDR
EQQLLHQKPKDVITIKQLPLAGSNNNNINNNINNNNNNNNIMEDPMADT

A nucleic acid sequence encoding the insect, preferably Drosophila melanogaster, tyrosylprotein sulfotransferase may comprise SEQ ID No. 24, whereby nucleotide residues 1 to 180, preferably 1 to 165, more preferably 1 to 150, more preferably 1 to 147, more preferably 1 to 144, more preferably 1 to 138, more preferably 1 to 135, more preferably 1 to 120, more preferably 1 to 105, in particular 1 to 141, of SEQ ID No. 24 encode the CTS region:

(SEQ ID No. 24)
atgcgactgccatatcgaaataagaaggtcaccctgtgggtgctcttcgg
catcatcgtcatcaccatgttcctattcaaattcaccgaactgcggccca
catgcctcttcaaggtggacgccgccaacgagctctcctcccaaatggtt
cgcgttgagaaatacctcacagatgacaatcaacgcgtttattcatacaa
ccgtgagatgccattaatattcataggcggcgtgccgagatctgggacga
ctttgatgcgcgccatgctggatgcccatcccgatgtgcgctgcgggcag
gaaacccgtgtcattccgcgcatcctgcagctgcgctcgcactggctgaa
gtccgagaaggagtcgctccgcctgcaggaggccggcatcaccaaagagg
tcatgaacagtgccatcgcgcagttctgtctggaaatcatcgccaaacac
ggcgagccggcgccgcgcttatgcaacaaggatccgctgacgctgaaaat
gggctcctatgtcatcgagctatttccgaacgctaaattcctattcatgg
tgcgcgacggccgggcgacagttcattcgattatatcgcgcaaggtgaca
atcaccggcttcgatttgagcagctaccggcagtgcatgcagaagtggaa
ccacgccatcgaggtgatgcacgagcagtgccgggacatcggcaaggacc
gctgcatgatggtttactatgagcagctggtactgcatcccgaggagtgg
atgcgaaagatactgaaattcctggacgtgccatggaacgatgcggtgct
gcaccacgaggagttcataaataaaccgaacggtgtgcctctgtccaagg
tggaacgttcgtcggaccaggttatcaagccggttaatctggaggcgatg
tccaaatgggttggccaaatacccggcgacgtggtgcgcgacatggccga
catagcgcccatgctgtccgtgctcggctacgatccgtacgcgaatccgc
cggactatggtaagccagatgcatgggtgcaggacaacacgtcgaagtta
aaggccaatcgaatgctgtgggagagtaaggcgaagcaagtgctgcagat
gtcatccagcgaggatgacaacacgaacaccatcatcaacaatagcaaca
ataaggataacaacaataatcagtacacaatcaataaaattataccagaa
caacacagcagacagcggcaacatgtacagcagcaacatctgcagcagca
gcagcagcagcatctgcaacagcagcaacatcagcggcagcagcaacagc
agcaacgtgaggaggagagcgagtcggaaagggaagcggaaccggatcga
gaacaacaattgttgcatcaaaagccaaaggatgtcattacgataaagca
gctgccattagctgggagcaacaataacaacatcaacaataacatcaaca
acaacaacaacaacaacaacatcatggaggaccccatggcggatacatga

Particularly preferred is a plant tyrosylprotein sulfotransferase of Arabidopsis thaliana comprising amino acid sequence SEQ ID No. 25, whereby amino acid residues 1 to 24 of SEQ ID No. 25 represent a signal sequence and amino acid residues 450 to 500, preferably 460 to 500, more preferably 465 to 500, more preferably 469 to 500, more preferably 470 to 500, in particular 471 to 500, of SEQ ID No. 25 represent the TS region (transmembrane domain with cytosolic tail; see e.g. Moore, PNAS 106(2009):14741-2):

(SEQ ID No. 25)
MQMNSVWKLSLGLLLLSSVIGSFAELDFGHCETLVKKWADSSSSREEHVN
KDKRSLKDLLFFLHVPRTGGRTYFHCFLRKLYDSSEECPRSYDKLHFNPR
KEKCKLLATHDDYSLMAKLPRERTSVMTIVRDPIARVLSTYEFSVEVAAR
FLVHPNLTSASRMSSRIRKSNVISTLDIWPWKYLVPWMREDLFARRDARK
LKEVVIIEDDNPYDMEEMLMPLHKYLDAPTAHDIIHNGATFQIAGLTNNS
HLSEAHEVRHCVQKFKSLGESVLQVAKRRLDSMLYVGLTEEHRESASLFA
NVVGSQVLSQVVPSNATAKIKALKSEASVTISETGSDKSNIQNGTSEVTL
NKAEAKSGNMTVKTLMEVYEGCITHLRKSQGTRRVNSLKRITPANFTRGT
RTRVPKEVIQQIKSLNNLDVELYKYAKVIFAKEHELVSNKLISSSKRSIV
DLPSELKSVLGEMGEEKLWKFVPVALMLLLIVLFFLFVNAKRRRTSKVKI

A nucleic acid sequence encoding the plant, preferably Arabidopsis thaliana, tyrosylprotein sulfotransferase may comprise SEQ ID No. 26, whereby nucleotide residues 1 to 72 of SEQ ID No. 26 represent a signal sequence and nucleotide residues 1350 to 1500, preferably 1380 to 1500, more preferably 1395 to 1500, more preferably 1407 to 1500, more preferably 1410 to 1500, in particular 1413 to 1500, of SEQ ID No. 25 represent the TS region (transmembrane domain with cytosolic tail; see e.g. Moore, PNAS 106(2009):14741-2):

(SEQ ID No. 26)
ATGCAAATGAACTCTGTTTGGAAGCTGTCTCTTGGGTTATTACTTCTTAG
CTCAGTTATTGGCTCTTTTGCGGAACTTGATTTTGGCCATTGCGAAACTC
TTGTGAAAAAATGGGCTGATTCTTCTTCATCTCGTGAAGAACATGTTAAT
AAAGACAAACGCTCGCTTAAGGATTTGCTCTTCTTTCTCCACGTTCCGCG
AACTGGAGGCAGAACATATTTTCATTGTTTTTTGAGGAAGTTGTATGATA
GCTCTGAGGAATGTCCTCGATCTTACGACAAGCTCCACTTCAATCCAAGG
AAGGAAAAGTGCAAGTTGTTAGCCACACATGATGATTATAGTTTGATGGC
AAAGCTTCCGAGGGAGAGAACTTCGGTGATGACAATAGTTCGGGATCCTA
TTGCGCGTGTGTTAAGCACTTATGAATTTTCCGTAGAGGTAGCAGCTAGG
TTTTTGGTGCATCCCAATTTAACTTCTGCGTCAAGGATGTCTAGCCGCAT
ACGCAAGAGTAATGTAATAAGCACACTAGACATATGGCCATGGAAATACC
TAGTTCCATGGATGAGAGAAGACTTGTTTGCTCGGCGAGATGCACGAAAA
TTGAAGGAGGTAGTGATCATTGAGGACGATAACCCGTATGACATGGAGGA
GATGCTTATGCCTTTGCACAAATATCTTGATGCGCCTACTGCTCATGACA
TCATCCACAATGGAGCGACTTTTCAGATTGCAGGATTGACAAATAACTCC
CATTTATCAGAAGCACACGAGGTTCGGCATTGTGTGCAGAAATTCAAAAG
CCTTGGTGAGTCTGTTCTCCAAGTTGCCAAGAGGAGGCTAGACAGCATGT
TGTATGTTGGACTGACAGAGGAGCACAGGGAATCTGCATCACTTTTTGCC
AATGTAGTGGGTTCTCAAGTGCTGTCTCAAGTGGTTCCGTCCAATGCAAC
TGCGAAAATCAAAGCTCTTAAATCAGAAGCAAGTGTCACAATTTCAGAAA
CCGGGTCAGATAAGAGTAATATTCAGAATGGTACATCTGAAGTTACATTG
AATAAGGCAGAAGCTAAGAGTGGGAATATGACGGTAAAAACCCTTATGGA
AGTCTATGAAGGCTGCATCACTCATTTACGAAAGTCCCAAGGAACCAGAC
GGGTCAACTCTCTGAAGAGAATAACTCCAGCAAATTTTACAAGAGGGACG
CGTACAAGAGTTCCTAAAGAGGTCATTCAGCAGATCAAATCGCTTAACAA
CCTCGATGTGGAGCTCTACAAATATGCAAAAGTAATCTTTGCCAAAGAAC
ATGAATTAGTGTCGAATAAGTTGATCTCAAGTTCTAAGAGAAGCATTGTT
GATCTGCCGAGTGAGTTAAAGAGCGTATTGGGAGAAATGGGTGAAGAGAA
GCTATGGAAGTTCGTACCAGTGGCATTGATGCTTTTATTGATCGTCCTCT
TCTTTCTATTTGTAAACGCTAAAAGGAGAAGAACCTCCAAAGTTAAGATT
TGA

If a tyrosylprotein sulfotransferase comprising a TS region at its C-terminus (e.g. SEQ ID No. 25) is used according to the present invention, the TS region is preferably removed and the signal peptide located at the N-terminus is preferably exchanged with a heterologous CTS regions.

According to a preferred embodiment of the present invention the catalytically active fragment of a tyrosylprotein sulfotransferase comprises or consists of an amino acid sequence selected from the group consisting of amino acid residues 31 to 370, preferably 36 to 370, more preferably 38 to 370, more preferably 39 to 370, more preferably 41 to 370, more preferably 42 to 370, more preferably 43 to 370, more preferably 46 to 370, more preferably 51 to 370, in particular 40 to 370, of SEQ ID No. 1; amino acid residues 31 to 377, preferably 36 to 377, more preferably 39 to 377, more preferably 40 to 377, more preferably 42 to 377, more preferably 43 to 377, more preferably 46 to 377, more preferably 51 to 377, in particular 41 to 377, of SEQ ID No. 13; amino acid residues 31 to 370, preferably 36 to 370, more preferably 38 to 370, more preferably 39 to 370, more preferably 41 to 370, more preferably 42 to 370, more preferably 43 to 370, more preferably 46 to 370, more preferably 51 to 370, in particular 40 to 370, of SEQ ID No. 15; amino acid residues 41 to 390, preferably 46 to 390, more preferably 51 to 390, more preferably 53 to 390, more preferably 55 to 390, more preferably 56 to 390, more preferably 61 to 390, more preferably 66 to 390, in particular 54 to 390, of SEQ ID No. 16; amino acid residues 31 to 380, preferably 36 to 380, more preferably 38 to 380, more preferably 39 to 380, more preferably 41 to 380, more preferably 42 to 380, more preferably 43 to 380, more preferably 46 to 380, more preferably 51 to 380, in particular 40 to 380, of SEQ ID No. 19; amino acid residues 31 to 259, preferably 36 to 259, more preferably 39 to 259, more preferably 41 to 259, more preferably 43 to 259, more preferably 46 to 259, more preferably 51 to 259, of SEQ ID No. 20; amino acid residues 36 to 499, preferably 41 to 499, more preferably 46 to 499, more preferably 47 to 499, more preferably 49 to 499, more preferably 50 to 499, more preferably 51 to 499, more preferably 56 to 499, more preferably 61 to 499, in particular 48 to 499, of SEQ ID No. 23; and amino acid residues 449 to 500, preferably 25 to 459, more preferably 25 to 464, more preferably 25 to 468, more preferably 25 to 469, in particular 25 to 470, of SEQ ID No. 25.

According to a further preferred embodiment of the present invention the heterologous CTS region comprises a nucleic acid sequence selected from the group consisting of SEQ ID No. 3, SEQ ID No. 5 and SEQ ID No. 7.

The heterologous CTS region derived from a human tyrosylprotein sulfotransferase comprises preferably amino acid sequence SEQ ID No. 3:

MVGKLKQNLLLACLVISSVTVFYLGQHAMECHHRIEERS

SEQ ID No. 3 may be encoded by nucleic acid sequence SEQ ID No. 4:

atggttggaaagctgaagcagaacttactattggcatgtctggtgatt
agttctgtgactgtgttttacctgggccagcatgccatggaatgccat
caccggatagaggaacgtagc

The heterologous CTS region derived from α1,3Fucosyltransferase 11 of Arabidopsis thaliana comprises preferably amino acid sequence SEQ ID No. 5 (Met¹-Val⁶⁸ of AEE76217.1):

MGVFSNLRGPKIGLTHEELPVVANGSTSSSSSPSSFKRKVSTFLPICV
ALVVIIEIGFLCRLDNAS

SEQ ID No. 5 may be encoded by nucleic acid sequence SEQ ID No. 6:

atgggtgttttctccaatcttcgaggtcctaaaattggattgacccat
gaagaattgcctgtagtagccaatggctctacttcttcttcttcgtct
ccttcctctttcaagcgtaaagtctcgacctttttgccaatctgcgtg
gctcttgtcgtcattatcgagatcgggttcctctgtcggctcgataac
gcttct

The heterologous CTS region derived from α2,6sialytransferase of Rattus norvegicus comprises preferably amino acid sequence SEQ ID No. 7 (Met¹-Gly⁵⁴ of M18769.1):

MIHTNLKKKFSLFILVFLLFAVICVWKKGSDYEALTLQAKEFQMPKSQ
EKVA

SEQ ID No. 7 may be encoded by nucleic acid sequence SEQ ID No. 8:

atgattcataccaacttgaagaaaaagttcagcctcttcatcctggtc
tttctcctgttcgcagtcatctgtgtttggaagaaagggagcgactat
gaggcccttacactgcaagccaaggaattccagatgcccaagagccag
gagaaagtggcc

The nucleic acid according to the present invention may encode a human tyrosylprotein sulfotransferase whose wild-type CTS region at its N-terminus has been replaced by a CTS region of α1,3-Fucosyltransferase 11 of Arabidopsis thaliana and may comprise or consist of SEQ ID No. 9:

atgggtgttttctccaatcttcgaggtcctaaaattggattgacccat
gaagaattgcctgtagtagccaatggctctacttcttcttcttcgtct
ccttcctctttcaagcgtaaagtctcgacctttttgccaatctgcgtg
gctcttgtcgtcattatcgagatcgggttcctctgtcggctcgataac
gcttct(n)₀₋₁₀ cagccagtcaaattggagagcacaaggaccactgtg
agaactggcctggacctcaaagccaacaaaacctttgcctatcacaaa
gatatgcctttaatatttattggaggtgtgcctcggagtggaaccaca
ctcatgagggccatgctggacgcacatcctgacattcgctgtggagag
gaaaccagggtcattccccgaatcctggccctgaagcagatgtggtca
cggtcaagtaaagagaagatccgcctggatgaggctggtgttactgat
gaagtgctggattctgccatgcaagccttcttactagaaattatcgtt
aagcatggggagccagccccttatttatgtaataaagatccttttgcc
ctgaaatctttaacttacctttctaggttattccccaatgccaaattt
ctcctgatggtccgagatggccgggcatcagtacattcaatgatttct
cgaaaagttactatagctggatttgatctgaacagctatagggactgt
ttgacaaagtggaatcgtgctatagagaccatgtataaccagtgtatg
gaggttggttataaaaagtgcatgttggttcactatgaacaacttgtc
ttacatcctgaacggtggatgagaacactcttaaagttcctccagatt
ccatggaaccactcagtattgcaccatgaagagatgattgggaaagct
gggggagtgtctctgtcaaaagtggagagatctacagaccaagtaatc
aagccagtcaatgtaggagctctatcaaaatgggttgggaagataccg
ccagatgttttacaagacatggcagtgattgctcctatgcttgccaag
cttggatatgacccatatgccaacccacctaactacggaaaacctgat
cccaaaattattgaaaacactcgaagggtctataagggagaattccaa
ctacctgactttcttaaagaaaaaccacagactgagcaagtggagtag

The nucleic acid molecule encoding a modified tyrosylprotein sulfotransferase comprising or consisting of SEQ ID No. 9 may comprise a linker between the nucleic acid stretch encoding a heterologous CTS region (italic) and the nucleic acid stretch encoding a tyrosylprotein sulfotransferase. This linker may consists of 1 to 10, preferably 2 to 9, more preferably 3 to 8, more preferably 4 to 7, in particular 6, nucleotides (n). This linker can be a result of fusing the heterologous CTS region to the tyrosylprotein sulfotransferase and thus comprise restriction sites or may have other functions. In a particularly preferred embodiment of the present invention the linker consists of or comprises the nucleic acid sequence GGATCC.

The nucleic acid according to the present invention may encode a human tyrosylprotein sulfotransferase whose wild-type CTS region at its N-terminus has been replaced by a CTS region of an α2,6-sialytransferase of Rattus norvegicus and may comprise or consist of SEQ ID No. 10:

atgattcataccaacttgaagaaaaagttcagcctcttcatcctggtct
ttctcctgttcgcagtcatctgtgtttggaagaaagggagcgactatga
ggcccttacactgcaagccaaggaattccagatgcccaagagccaggag
aaagtggcc(n)₀₋₁₀ cagccagtcaaattggagagcacaaggaccactg
tgagaactggcctggacctcaaagccaacaaaacctttgcctatcacaa
agatatgcctttaatatttattggaggtgtgcctcggagtggaaccaca
ctcatgagggccatgctggacgcacatcctgacattcgctgtggagagg
aaaccagggtcattccccgaatcctggccctgaagcagatgtggtcacg
gtcaagtaaagagaagatccgcctggatgaggctggtgttactgatgaa
gtgctggattctgccatgcaagccttcttactagaaattatcgttaagc
atggggagccagccccttatttatgtaataaagatccttttgccctgaa
atctttaacttacctttctaggttattccccaatgccaaatttctcctg
atggtccgagatggccgggcatcagtacattcaatgatttctcgaaaag
ttactatagctggatttgatctgaacagctatagggactgtttgacaaa
gtggaatcgtgctatagagaccatgtataaccagtgtatggaggttggt
tataaaaagtgcatgttggttcactatgaacaacttgtcttacatcctg
aacggtggatgagaacactcttaaagttcctccagattccatggaacca
ctcagtattgcaccatgaagagatgattgggaaagctgggggagtgtct
ctgtcaaaagtggagagatctacagaccaagtaatcaagccagtcaatg
taggagctctatcaaaatgggttgggaagataccgccagatgttttaca
agacatggcagtgattgctcctatgcttgccaagcttggatatgaccca
tatgccaacccacctaactacggaaaacctgatcccaaaattattgaaa
acactcgaagggtctataagggagaattccaactacctgactttcttaa
agaaaaaccacagactgagcaagtggagtag

The nucleic acid molecule encoding a modified tyrosylprotein sulfotransferase comprising or consisting of SEQ ID No. 10 may comprise a linker between the nucleic acid stretch encoding a heterologous CTS region (italic) and the nucleic acid stretch encoding a tyrosylprotein sulfotransferase. This linker may consists of 1 to 10, preferably 2 to 9, more preferably 3 to 8, more preferably 4 to 7, in particular 6, nucleotides (n). This linker can be a result of fusing the heterologous CTS region to the tyrosylprotein sulfotransferase and thus comprise restriction sites or may have other functions. In a particularly preferred embodiment of the present invention the linker consists of or comprises the nucleic acid sequence CTCGAG.

Another aspect of the present invention relates to a polypeptide encoded by a nucleic acid molecule according to the present invention, preferably a polypeptide comprising or consisting of an amino acid sequence selected from the group consisting of SEQ ID No. 11 and 12.

The polypeptide according to the present invention may comprise or consist of a human tyrosylprotein sulfotransferase whose wild-type CTS region at its N-terminus has been replaced by a CTS region of an α1,3-Fucosyltransferase 11 of Arabidopsis thaliana and may comprise or consist of SEQ ID No. 11:

MGVFSNLRGPKIGLTHEELPVVANGSTSSSSSPSSFKRKVSTFLPICVA
LVVIIEIGFLCRLDNAS(X)₁₋₅ QPVKLESTRTTVRTGLDLKANKTFAY
HKDMPLIFIGGVPRSGTTLMRAMLDAHPDIRCGEETRVIPRILALKQMW
SRSSKEKIRLDEAGVTDEVLDSAMQAFLLEIIVKHGEPAPYLCNKDPFA
LKSLTYLSRLFPNAKFLLMVRDGRASVHSMISRKVTIAGFDLNSYRDCL
TKWNRAIETMYNQCMEVGYKKCMLVHYEQLVLHPERWMRTLLKFLQIPW
NHSVLHHEEMIGKAGGVSLSKVERSTDQVIKPVNVGALSKWVGKIPPDV
LQDMAVIAPMLAKLGYDPYANPPNYGKPDPKIIENTRRVYKGEFQLPDF
LKEKPQTEQVE

The modified tyrosylprotein sulfotransferase comprising or consisting of SEQ ID No. 11 may comprise a linker between the heterologous CTS region (italic) and the tyrosylprotein sulfotransferase. This linker may consists of 1 to 5, preferably 1 to 4, more preferably 2 to 5, more preferably 2 or 3, in particular 2, amino acid residues (X). This linker can be a result of fusing the heterologous CTS region to the tyrosylprotein sulfotransferase or may have other functions. In a particularly preferred embodiment of the present invention the linker consists of or comprises the amino acid sequence GS.

The polypeptide according to the present invention may comprise or consist of a human tyrosylprotein sulfotransferase whose wild-type CTS region at its N-terminus has been replaced by a CTS region of an α2,6-sialytransferase of Rattus norvegicus and may comprise or consist of SEQ ID No. 12:

MIHTNLKKKFSLFILVFLLFAVICVWKKGSDYEALTLQAKEFQMPKSQE
KVA(X)₁₋₅ QPVKLESTRTTVRTGLDLKANKTFAYHKDMPLIFIGGVPR
SGTTLMRAMLDAHPDIRCGEETRVIPRILALKQMWSRSSKEKIRLDEAG
VTDEVLDSAMQAFLLEIIVKHGEPAPYLCNKDPFALKSLTYLSRLFPNA
KFLLMVRDGRASVHSMISRKVTIAGFDLNSYRDCLTKWNRAIETMYNQC
MEVGYKKCMLVHYEQLVLHPERWMRTLLKFLQIPWNHSVLHHEEMIGKA
GGVSLSKVERSTDQVIKPVNVGALSKWVGKIPPDVLQDMAVIAPMLAKL
GYDPYANPPNYGKPDPKIIENTRRVYKGEFQLPDFLKEKPQTEQVE

The modified tyrosylprotein sulfotransferase comprising or consisting of SEQ ID No. 10 may comprise a linker between the heterologous CTS region (italic) and the tyrosylprotein sulfotransferase. This linker may consists of 1 to 5, preferably 1 to 4, more preferably 2 to 5, more preferably 2 or 3, in particular 2, amino acid residues (X). This linker can be a result of fusing the heterologous CTS region to the tyrosylprotein sulfotransferase or may have other functions. In a particularly preferred embodiment of the present invention the linker consists of or comprises the amino acid sequence LE.

The term “polypeptide” is used herein interchangeably with the term “protein” and both terms refer to a polymer of amino acid residues.

Another aspect of the present invention relates to a vector comprising a nucleic acid molecule according to the present invention.

The vector of the present invention can be used to clone the nucleic acid molecule of the present invention, as a shuttle or as an expression vector in host cells. Expression vectors may include an expression cassette which comprises various specified nucleic acid elements which permit transcription of a particular nucleic acid in a host cell. Typically, expression cassettes comprise, among other sequences, a nucleic acid to be transcribed, a promoter and a terminator.

The vector of the present invention can be designed to be integrated into the genome of the host cell. Alternatively the vector is designed to not integrate into the genome of a host cell allowing transient expression of the nucleic acid molecule of the present invention. In the latter case the vector remains in a non-integrated state free within the cell.

A “vector” according to the present invention refers to a nucleic acid used to introduce the nucleic acid molecule of the present invention into a host cell. Expression vectors permit transcription of a nucleic acid inserted therein.

In order to enable a host cell to express polypeptide of the present invention encoded by the nucleic acid molecule as defined above the vector of the present invention comprises a promoter operably linked to the nucleic acid molecule.

As used herein, “operably linked” refers to a functional linkage between a promoter and the nucleic acid molecule encoding the polypeptide of the present invention, wherein the promoter sequence initiates and mediates transcription of the DNA sequence corresponding to said nucleic acid molecule.

The term “promoter”, as used herein, refers to a region of a nucleic acid molecule upstream from the start of transcription and involved in recognition and binding of RNA polymerase and other proteins to initiate transcription. Promoters are able to control (initiate) transcription in a cell. Plant promoters are able of initiating transcription in plant cells whether or not its origin is a plant cell. Such promoters include promoters obtained from plants, plant viruses and bacteria which comprise genes expressed in plant cells such Agrobacterium or Rhizobium. The promoter used in the vector of the present invention can be “inducible” or “repressible”, i.e. under environmental control. Such promoters can be controlled by changing the cultivation conditions (e.g. temperature) or by adding specific substances. Of course, the promoter used in the vectors of the present invention may be a “constitutive” promoter. Constitutive promoters are active under most environmental conditions and express continuously a protein or polypeptide of interest.

According to a preferred embodiment of the present invention the promoter is selected from the group consisting of promoters active in plants and plant cells, like the cauliflower mosaic virus 35S promoter, opine (octopine, nopaline, etc.) synthase promoters, actin promoter, ubiquitine promoter, etc.

In order to prevent transcriptional activation of downstream nucleic acid sequences by upstream promoters the vector of the present invention may comprise a “terminator” or “terminator sequence”. According to a preferred embodiment of the present invention the vector comprises a terminator which is preferably a g7T terminator. Another aspect of the present invention relates to a host cell comprising a nucleic acid molecule or a vector according to the present invention.

A “host cell”, as used herein, refers to a cell that contains a nucleic acid molecule or a vector and supports the replication and/or expression of said nucleic acid molecule or said vector. Host cells may be prokaryotic cells such as E. coli or eukaryotic cells such as yeast, plant, insect, amphibian or mammalian cells. In a particular preferred embodiment of the present invention the host cell is a plant cell. For cloning purposes and/or for producing a nucleic acid molecule of the present invention it is preferred to use prokaryotic cells, in particular E. coli. It is particularly preferred to use plant cells as host cells.

Another aspect of the present invention relates to a plant or plant cell capable of transferring a sulfate moiety to a tyrosine residue of a polypeptide which is a substrate of a tyrosylprotein sulfotransferase and preferably of animal origin heterologously produced in said plant or plant cell comprising a nucleic acid molecule or a vector according to the present invention.

A “substrate of a tyrosylprotein sulfotransferase” is any polypeptide or protein which can be sulfated in the presence of a tyrosylprotein sulfotransferase and a sulfate donor like phosphoadenosine-5′-phosphosulfate. Such substrates can be identified, for instance, by contacting and incubating them with a tyrosylprotein sulfotransferase and a sulfate donor (see e.g. Seibert C et al (Biochemistry 47(2008): 11251-11262)). Particularly preferred substrates are polypeptides and proteins of animal origin. Exemplary polypeptides which can be used as substrate for a tyrosylprotein sulfotransferase include PG9, PG16, hirudin and gp120.

“Polypeptides of animal origin”, as used herein, refers to polypeptides which are not naturally occurring in plants and plant cells or any other non-animal organism. Polypeptides of animal origin can be derived from the genome of animals and are usually expressed in animals. However, polypeptides derived from non-animal organisms which are homologs of animal derived polypeptides are not encompassed by this definition.

The term “plant cell”, as used herein, refers to protoplasts, gamete producing cells and cells which regenerate into whole plants. Plant cells, as used herein, further include cells obtained from or found in seeds, suspension cultures, embryos, meristematic regions, callus tissue, leaves, roots, shoots, gametophytes, sporophytes, pollen and microspores.

The plant and plant cell of the present invention may carry the nucleic acid molecule of the present invention in a nonintegrated form (e.g. as a vector) or may be a “transgenic plant” or “transgenic plant cell”. Such a plant or plant cell comprises within its genome a heterologous nucleic acid molecule. This heterologous nucleic acid molecule is usually stably integrated within the genome such that the polynucleotide is passed on to successive generations. In order to allow the plant and plant cell to express the polypeptide of the present invention, its encoding nucleic acid molecule is operably linked to a promoter.

The nucleic acid molecule and the vector according to the present invention enable a plant or plant cell to sulfate polypeptides and proteins expressed in said plant or plant cell. Therefore, the transgenic plant or plant cell of the present invention comprise preferably a nucleic acid molecule encoding a heterologous polypeptide operably linked to a promoter region.

A “heterologous protein” or “heterologous polypeptide”, as defined herein, refers to a protein or polypeptide that is not expressed by the plant or plant cell in nature. This is in contrast with a homologous protein which is a protein naturally expressed by a plant or plant cell. The heterologous expression of polypeptides and proteins within a host cell can be achieved by means and methods known in the art and described above for the polypeptide of the present invention.

Exemplary plants to be used according to the present invention include Nicotiana spp, Arabidopsis thaliana, Algae, duckweed (Lemna minor), mosses (e.g. Physcomitrella), corn (Zea mays), rice (Oryza sativa), wheat (Triticum), peas (Pisum sativum), flax (Linum usitatissimum) and rapeseed (Brassica napus).

According to a preferred embodiment of the present invention the heterologous polypeptide of animal origin is a mammalian, more preferably human, polypeptide.

According to a further embodiment of the present invention the heterologous animal polypeptide is an antibody.

Examples of heterologous polypeptides and proteins that can be advantageously produced in a sulfated form by the plants or plant cells of the present invention include antibodies or fragments thereof which are selected from the group consisting of monoclonal antibodies, chimeric antibodies, humanized antibodies, human antibodies, multispecific antibodies, Fab, Fab′, F(ab′)2, Fv, domain antibody (dAb), complementarity determining region (CDR) fragments, CDR-grafted antibodies, single-chain antibodies (ScFv), single chain antibody fragments, chimeric antibodies, diabodies, triabodies, tetrabodies, minibodies, linear antibodies, chelating recombinant antibodies, tribodies, bibodies, intrabodies, nanobodies, small modular immunopharmaceuticals (SMIP), camelized antibodies, VHH containing antibodies and polypeptides that comprise at least a portion of an immunoglobulin that is sufficient to confer specific antigen binding to the polypeptide, such as one, two, three, four, five or six CDR sequences.

According to a particularly preferred embodiment of the present invention the antibody is an antibody binding to an HIV surface protein.

As used herein, the term “specific for” can be used interchangeably with “binding to” or “binding specifically to”. These terms characterize molecules that bind to an antigen or a group of antigens with greater affinity (as determined by, e.g., ELISA or BlAcore assays) than other antigens or groups of antigens. According to the present invention molecules “specific for” an antigen may also be able to bind to more than one, preferably more than two, more preferably more than three, even more preferably more than five, antigens. Such molecules are defined to be “cross-reactive” (e.g. cross-reactive immunoglobulins, crossreactive antigen binding sites).

The antibody expressed and sulfated by the plant cell and plant of the present invention is preferably an antibody selected from the group consisting of PG9, PG16, 47e, 412d, Sb1, C12, E51, CM51 and a variant thereof.

It is known that sulfated antibodies may have a greater binding affinity to an antigen compared to its non-sulfated version. This is effect is particularly significant for antibodies like PG9, PG16 (see e.g. Pejchal R at al. Proc Natl Acad Sci USA 107(2010):11483-11488),PGT141-145 (see e.g. McLellan J S et al. Nature 480(2011):336-343), 47e, 412d, Sb1, C12, E51, CM51 (see e.g. Choe H et al. Cell 114(2003):161-170 and Huang C C et al. Proc Natl Acad Sci USA 101(2004):2706-2711) and variants thereof. Therefore, it is particularly advantageous to provide efficient tools for producing sulfated PG9, PG16, PGT141-145, 47e, 412d, Sb1, C12, E51, CM51 and variants thereof.

According to a preferred embodiment of the present invention the antibody variant is selected from the group consisting of PG9 comprising modifications at RL94SHL95A.

According to a further embodiment of the present invention the plant is Nicotiana benthamiana, Nicotiana spp, Arabidopsis thaliana, Algae, Lemna minor, Physcomitrella, Zea mays, Oryza sativa, Triticum, Pisum sativum, Linum usitatissimum or Brassica napus, whereby Nicotiana benthamiana is particularly preferred.

Another aspect of the present invention relates to a method of recombinantly producing a polypeptide of animal origin carrying an animal-type sulfation comprising the step of cultivating a plant or plant cell according to the present invention.

Methods and means to cultivate recombinant plants and plant cells are known in the art.

The present invention is further illustrated by the following figures and examples without being restricted thereto.

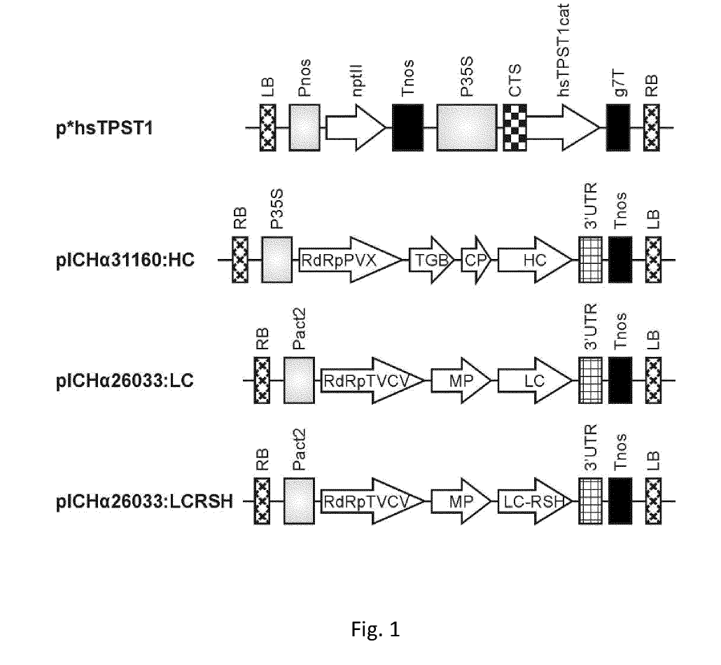

FIG. 1 shows the PG9 and TPST1 constructs used in the examples. PG9HC, PG9LC and PG9LC-RSH were cloned into Magnlcon vectors. Three versions of hsTPST1 containing different CTS regions were cloned into pPT2 giving rise to p^{Full}hsTPST1, p^{RST}hsTPST1 and p^{Full}hsTPST1.

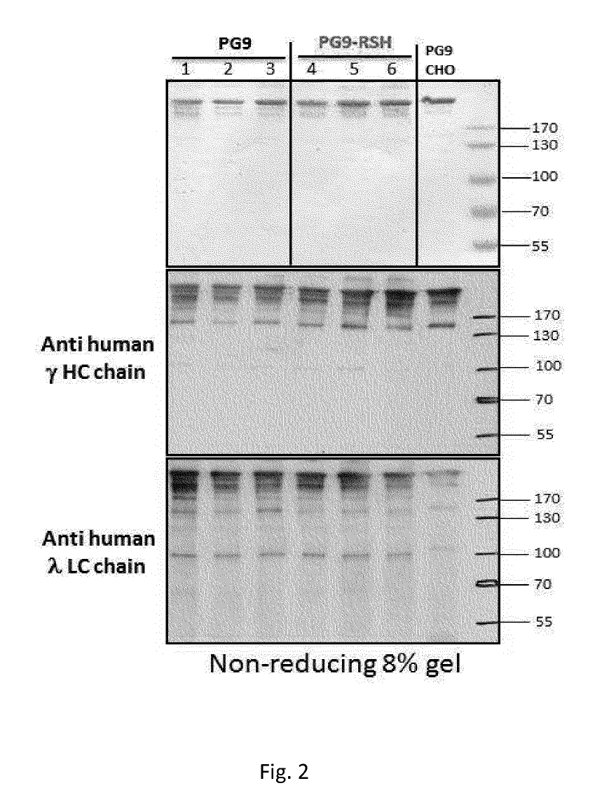

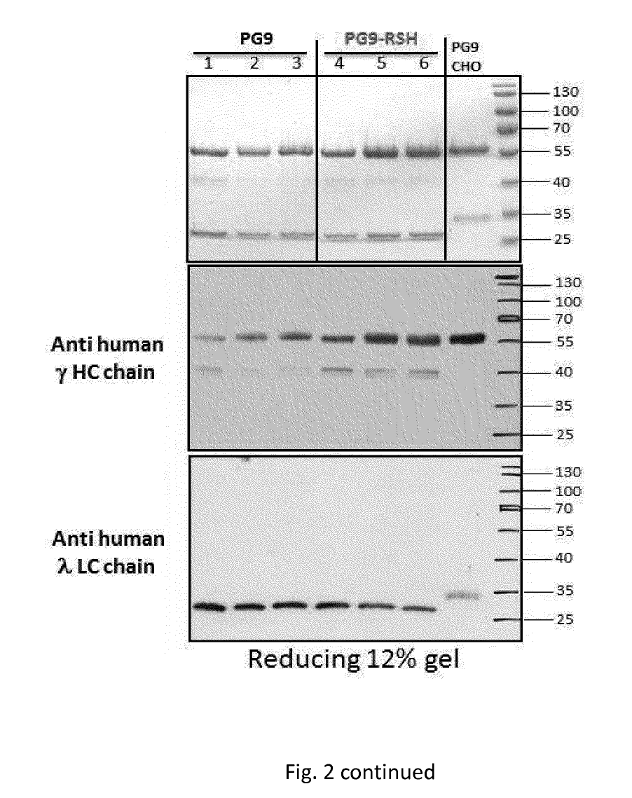

FIG. 2 shows the purification of plant-produced PG9 and RSH and comparison to ^{CHO}PG9. Coomassie staining (top panels) and immunoblotting (middle and bottom panels) of purified PG9 and RSH after separation by SDS-PAGE under reducing (right) and non-reducing (left) conditions. 1: ^{ΔXF}PG9; 2: ^{ΔXF}PG9_{sulf}; 3: ^{ΔXF}PG9_{sulfsia}; 4: ^{ΔXF}RSH; 5: ^{ΔXF}RSH_{Sulf}; 6: ^{ΔXF}RSH_{sulfsia}.

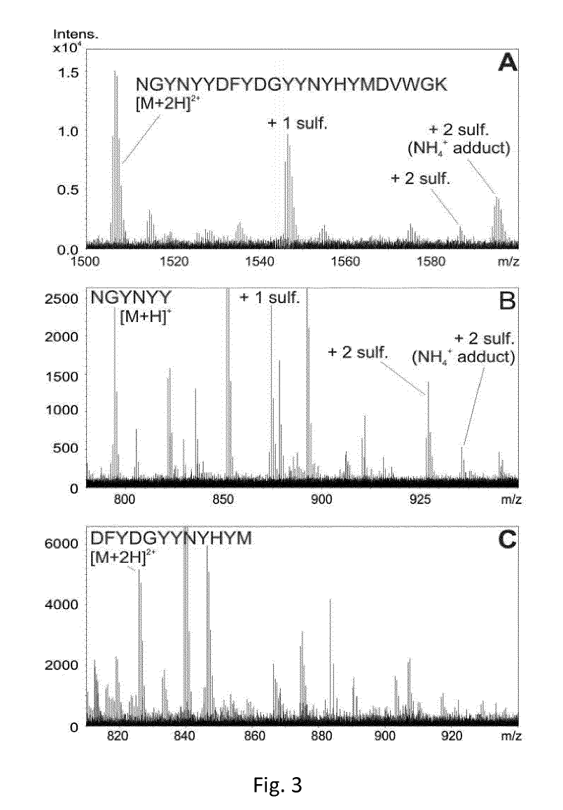

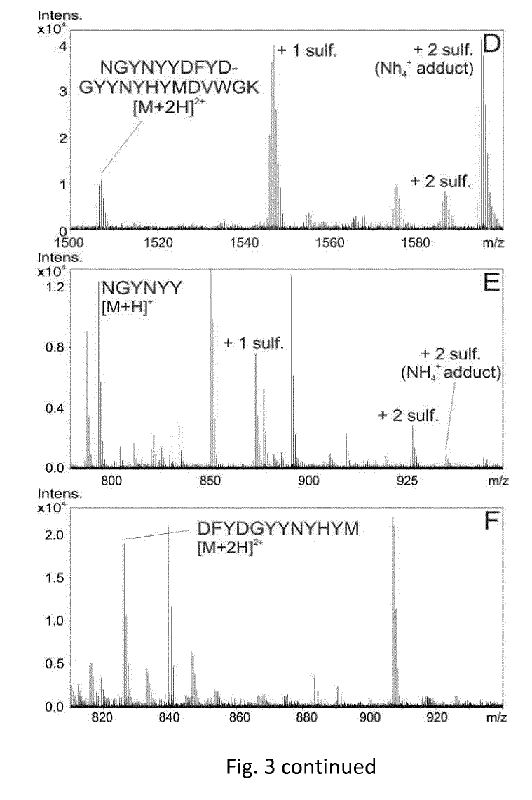

FIG. 3 shows ^{ΔXF}PG9 and ^{CHO}PG9 are singly and doubly sulfated on tyrosine residues Y^{100E}, Y^{100G} or Y^{100H}. The sulfation sites of ^{ΔXF}PG9 and ^{CHO}PG9 were mapped by LC-ESI-MS to the tryptic PG9 peptide N^{100C}GYNYYDFYDGYYNYHYMDVWGK¹⁰⁵ (SEQ ID No. 27) (panels A and D, respectively). Further digestion with AspN revealed nonsulfated, singly and doubly sulfated variants of the peptide N^{100C}GYNYY^{100H} (SEQ ID No. 28) (panels B and E for ^{ΔXF}PG9 and ^{CHO}PG9). No sulfated residues were found on the other AspN fragment, D¹⁰⁰¹FYDGYYNYHYM^{100T} (SEQ ID No. 29) (panels C and F for ^{ΔXF}PG9 and ^{CHO}PG9).

### EXAMPLES

### Materials and Methods

1. Cloning of Neutralizing Anti-HIV Antibody PG9 and its Variant RSH

The signal peptide of barley a-amylase (amino acid residues 1 to 24 of the amino acid sequence of acc. no. CAX51374.1) was cloned into Magnlcon vectors pICH26033 and pICH31160 (Niemer, M., et al. Biotechn J 9(2014):493-500) to give rise to pICHα26033 and pICHα31160. cDNA codon-optimized for Nicotiana benthamiana and encoding a Bsal site followed by the PG9 (McLellan J S et al. Nature 480(2011):336-343; Protein Data Bank accession nos. 3U36_A, 3U36_B, 3U36_C, 3U36_D, 3U36_E, 3U36_F) variable heavy and C_{H}1 domain without signal peptide was PCR-amplified.

PG9LC (SEQ ID No. 30; PDB-database: 3U2S, 3U36, 3U4E, 3MUH) and PG9LC-RSH (SEQ ID No. 32) cDNAs (both encoding an N23Q mutation) were synthesized with codons optimized for Nicotiana benthamiana, without signal peptide but with 5′- and 3′ Bsal restriction sites.

PG9LC consists of amino acid sequence SEQ ID No. 30, whereby the signal peptide of barley a-amylase is marked in bold and italic:

[symbol]

[symbol]

QSALTQPASVSGSPGQSITISCQGT
SNDVGGYESVSWYQQHPGKAPKVVIYDVSKRPSGVSNRFSGSKSGNTAS
LTISGLQAEDEGDYYCKSLTSTRRRVFGTGTKLTVLGQPKAAPSVTLFP
PSSEELQANKATLVCLISDFYPGAVTVAWKADSSPVKAGVETTTPSKQS
NNKYAASSYLSLTPEQWKSHKSYSCQVTHEGSTVEKTVAPTECS

PG9LC is encoded by nucleotide sequence SEQ ID No. 31 (including stop codon), whereby the nucleic acid stretch encoding the signal peptide of barley a-amylase is marked in bold and italic:

[symbol]

[symbol]

[symbol]

[symbol]

[symbol]

CAGAGTGCTCTTACTCAGCCTGCTTC
TGTTTCTGGTTCTCCTGGTCAGAGCATCACCATTTCTTGCCAGGGAACC
TCTAACGATGTGGGAGGTTACGAGTCCGTGTCTTGGTATCAACAGCATC
CTGGTAAGGCTCCTAAGGTGGTGATCTACGATGTGAGCAAGAGGCCTTC
TGGTGTGAGCAATAGGTTCAGCGGTAGCAAGTCTGGTAACACCGCTTCT
CTTACCATCTCTGGACTTCAGGCTGAGGATGAGGGAGATTACTACTGCA
AGTCTCTGACCTCCACTAGAAGAAGGGTGTTCGGAACCGGTACTAAGCT
TACTGTTCTGGGTCAACCTAAGGCTGCTCCTTCTGTGACTTTGTTCCCT
CCATCTTCTGAGGAACTGCAGGCTAACAAGGCTACCCTTGTGTGCCTGA
TCAGCGATTTTTACCCTGGTGCTGTTACCGTGGCTTGGAAGGCTGATTC
TTCACCTGTTAAGGCTGGTGTGGAAACCACCACTCCTAGCAAGCAGAGC
AACAACAAGTACGCTGCTAGCTCCTACCTTAGCCTTACTCCTGAACAGT
GGAAGTCCCACAAGAGCTACTCATGCCAGGTTACCCATGAGGGTTCTAC
CGTGGAAAAGACTGTTGCTCCTACTGAGTGCAGCTAG

PG9LC-RSH consists of amino acid sequence SEQ ID No. 32, whereby the signal peptide of barley α-amylase is marked in bold and italic:

[symbol]

[symbol]

QSALTQPASVSGSPGQSITISCQGT
SNDVGGYESVSWYQQHPGKAPKVVIYDVSKRPSGVSNRFSGSKSGNTAS
LTISGLQAEDEGDYYCKSLTSRSHRVFGTGTKLTVLGQPKAAPSVTLFP
PSSEELQANKATLVCLISDFYPGAVTVAWKADSSPVKAGVETTTPSKQS
NNKYAASSYLSLTPEQWKSHKSYSCQVTHEGSTVEKTVAPTECS

PG9LC-RSH is encoded by nucleotide sequence SEQ ID No. 33 (including stop codon), whereby the nucleic acid stretch encoding the signal peptide of barley a-amylase is marked in bold and italic:

[symbol]

[symbol]

[symbol]

[symbol]

[symbol]

CAGAGTGCTCTTACTCAGCCTGCTTC
TGTTTCTGGTTCTCCTGGTCAGAGCATCACCATTTCTTGCCAGGGAACC
TCTAACGATGTGGGAGGTTACGAGTCCGTGTCTTGGTATCAACAGCATC
CTGGTAAGGCTCCTAAGGTGGTGATCTACGATGTGAGCAAGAGGCCTTC
TGGTGTGAGCAATAGGTTCAGCGGTAGCAAGTCTGGTAACACCGCTTCT
CTTACCATCTCTGGACTTCAGGCTGAGGATGAGGGAGATTACTACTGCA
AGTCTCTGACCTCCAGAAGTCACAGGGTGTTCGGAACCGGTACTAAGCT
TACTGTTCTGGGTCAACCTAAGGCTGCTCCTTCTGTGACTTTGTTCCCT
CCATCTTCTGAGGAACTGCAGGCTAACAAGGCTACCCTTGTGTGCCTGA
TCAGCGATTTTTACCCTGGTGCTGTTACCGTGGCTTGGAAGGCTGATTC
TTCACCTGTTAAGGCTGGTGTGGAAACCACCACTCCTAGCAAGCAGAGC
AACAACAAGTACGCTGCTAGCTCCTACCTTAGCCTTACTCCTGAACAGT
GGAAGTCCCACAAGAGCTACTCATGCCAGGTTACCCATGAGGGTTCTAC
CGTGGAAAAGACTGTTGCTCCTACTGAGTGCAGCTAG

PG9LC and PG9LC-RSH were inserted into pICHa26033 in frame after the barley a-amylase signal peptide. All vectors were transformed into E. coli by electroporation and upon sequence confirmation into the Agrobacterium tumefaciens strain GV3101pMP90.

2. Cloning of Tyrosylprotein Sulfotransferase (TPST) Constructs

For expression in plants, hsTPST1 (accession number AK313098.1, open reading frame from start to stop codon) was cloned into vector pPT2 (Strasser R et al. Biochem J 387(2005):385-391). Three different constructs containing different CTS regions were constructed (FIG. 1). p^{Full}hsTPST1 contains the authentic CTS region (SEQ ID No. 3), in p^{Fut11}hsTPST1 Met¹-Ser³⁹ of hsTPST1 is replaced by the CTS of A. thaliana Fut11 (Met¹-Val⁶⁸) (SEQ ID No. 5), and in p^{RST}hsTPST it is replaced by the CTS region of rat sialyltransferase (Met¹-Gly⁵⁴) (SEQ ID No. 7). The nucleic acid sequences encoding the respective polypeptides having SEQ ID No. 1, 11 and 12 consist of SEQ ID No. 2, 9 and 10, respectively. After transformation into E. coli and sequence confirmation, all constructs were transformed into Agrobacterium tumefaciens strain UIA143pMP90.

3. In Planta Expression of PG9 and RSH

N. benthamiana ΔXT/FT plants (age: 4-5 weeks) were used for co-infiltration with agrobacteria as described previously (Strasser R, et al. Plant Biotech J 6(2008):392-402). Briefly, liquid cultures of agrobacterial strains carrying pPG9HC, pPG9LC, pPG9LC-RSH, p^{Full}hsTPST1 , p^{Fut11}hsTPST1 and p^{RST}hsTPST1 were grown overnight, pelleted and resuspended in infiltration buffer (25 mM MES (pH 5.5), 25 mM MgSO₄, 0.1 mM acetosyringone). Mixtures of bacteria containing pPG9HC and pPG9LC (or pPG9LC-RSH) were infiltrated with or without different TPST constructs into N. benthamiana ΔXT/FT leaves. For in planta galactosylation and sialylation, an additional 6 genes had to be infiltrated (Castilho A et al. J Biol Chem 285(2010):15923-15930). OD₆₀₀ for infiltration was 0.01 for each of the IgG vectors, up to 0.8 for the TPST constructs and 0.05 for the vectors required for galactosylation/sialylation. Plants were harvested 3-6 days post infiltration.

4. Cloning, Expression and Purification of gp120^{ZM109}

The codon-optimized coding sequence (SEQ ID No. 34) for gp120 of HIV strain ZM109 (gp120¹⁰⁹) (SEQ ID No. 35) was appended with a C-terminal hexahistidine tag (bold and underlined in SEQ ID No. 34 and 35) and inserted into the HindIII/NotI sites of pCEP4 (Life Technologies).

gp120^{ZM109} (including a C-terminal histidine tag) is encoded by the nucleic acid sequence SEQ ID No. 34:

ATGCCTATGGGCAGCCTGCAGCCCCTGGCCACACTGTATCTGCTGGGAA
TGCTGGTGGCCAGCTGCCTGGGCGTGTGGAAAGAGGCCAAGACCACCCT
GTTCTGCGCCAGCGACGCCAAGAGCTACGAGCGCGAGGTGCACAATGTG
TGGGCCACCCATGCCTGCGTGCCCACCGATCCTGATCCCCAGGAACTCG
TGATGGCCAACGTGACCGAGAACTTCAACATGTGGAAGAACGACATGGT
GGACCAGATGCACGAGGACATCATCAGCCTGTGGGACCAGAGCCTGAAG
CCCTGCGTGAAGCTGACCCCTCTGTGCGTGACCCTGAACTGCACATCTC
CTGCCGCCCACAACGAGAGCGAGACAAGAGTGAAGCACTGCAGCTTCAA
CATCACCACCGACGTGAAGGACCGGAAGCAGAAAGTGAACGCCACCTTC
TACGACCTGGACATCGTGCCCCTGAGCAGCAGCGACAACAGCAGCAACA
GCTCCCTGTACAGACTGATCAGCTGCAACACCAGCACCATCACCCAGGC
CTGCCCCAAGGTGTCCTTCGACCCCATCCCCATCCACTACTGTGCCCCT
GCCGGCTACGCCATCCTGAAGTGCAACAACAAGACCTTCAGCGGCAAGG
GCCCCTGCAGCAACGTGTCCACCGTGCAGTGTACCCACGGCATCAGACC
CGTGGTGTCCACCCAGCTGCTGCTGAATGGCAGCCTGGCCGAAGAGGAA
ATCGTGATCAGAAGCGAGAACCTGACCGACAACGCCAAGACAATCATTG
TGCATCTGAACAAGAGCGTGGAAATCGAGTGCATCAGGCCCGGCAACAA
CACCAGAAAGAGCATCAGACTGGGCCCTGGCCAGACCTTTTACGCCACC
GGGGATGTGATCGGCGACATCCGGAAGGCCTACTGCAAGATCAACGGCA
GCGAGTGGAACGAGACACTGACAAAGGTGTCCGAGAAGCTGAAAGAGTA
CTTTAACAAGACCATTCGCTTCGCCCAGCACTCTGGCGGCGACCTGGAA
GTGACCACCCACAGCTTCAATTGCAGAGGCGAGTTCTTCTACTGCAATA
CCAGCGAGCTGTTCAACAGCAACGCCACCGAGAGCAATATCACCCTGCC
CTGCCGGATCAAGCAGATCATCAATATGTGGCAGGGCGTGGGCAGAGCT
ATGTACGCCCCTCCCATCCGGGGCGAGATCAAGTGCACCTCTAACATCA
CCGGCCTGCTGCTGACCAGGGACGGCGGAAACAACAACAATAGCACCGA
GGAAATCTTCCGGCCCGAGGGCGGCAACATGAGAGACAATTGGAGATCC
GAGCTGTACAAGTACAAGGTGGTGGAAATCAAGGGCCTGCGGGGCAGCC
ACCACCATCATCACCATTGA

gp120^{ZM109} (including a C-terminal histidine tag) consists of the amino acid sequence SEQ ID No. 35:

MPMGSLQPLATLYLLGMLVASCLGVWKEAKTTLFCASDAKSYEREVHNV
WATHACVPTDPDPQELVMANVTENFNMWKNDMVDQMHEDIISLWDQSLK
PCVKLTPLCVTLNCTSPAAHNESETRVKHCSFNITTDVKDRKQKVNATF
YDLDIVPLSSSDNSSNSSLYRLISCNTSTITQACPKVSFDPIPIHYCAP
AGYAILKCNNKTFSGKGPCSNVSTVQCTHGIRPVVSTQLLLNGSLAEEE
IVIRSENLTDNAKTIIVHLNKSVEIECIRPGNNTRKSIRLGPGQTFYAT
GDVIGDIRKAYCKINGSEWNETLTKVSEKLKEYFNKTIRFAQHSGGDLE
VTTHSFNCRGEFFYCNTSELFNSNATESNITLPCRIKQIINMWQGVGRA
MYAPPIRGEIKCTSNITGLLLTRDGGNNNNSTEEIFRPEGGNMRDNWRS
ELYKYKVVEIKGLRGSHHHHHH

Transient expression in FreeStyle™293F cells (Life Technologies) was performed following the instructions of the manufacturer. Culture supernatants were subjected to affinity chromatography using Ni²⁺-charged Chelating Sepharose (GE Healthcare), omitting the addition of phosphatase and protease inhibitors. Fractions eluted with 250 mM imidazole were dialyzed against PBS containing 0.02% (v/v) NaN₃ and then concentrated by ultrafiltration.

5. Monoclonal Antibody (mAb) Purification

Leaf material (see item 3.) infiltrated with the transformed Agrobacterium tumefaciens strains described under item 2 was crushed under liquid nitrogen, extracted twice for 20 min on ice with 45 mM Tris/HC1 (pH 7.4) containing 1.5 M NaCl, 40 mM ascorbic acid and 1 mM EDTA (2 ml per g leaf material) and cleared by centrifugation (4° C., 30 min, 27.500 g). Upon vacuum filtration through 10-μm cellulose filters (Roth, AP27.1) and centrifugation (4° C., 30 min, 27.500 g), the extract was filtered through a series of filters with pore sizes ranging from 10 μm to 0.2 μm (Roth, AP27.1, Roth, AP51.1, Roth, CT92.1, Roth, KH54.1) before being applied to a 1.5 ml Protein A Sepharose 4FF column (GE Healthcare, 17-1279-01) at 1 ml/min. Upon washing with PBS, bound mAbs were eluted with 100 mM glycine/HCl (pH 2.5). The eluate was immediately neutralized (1 M Tris/HCl (pH 8.0), 1.5 M NaCl), mAb-containing fractions were identified by their absorbance at 280 nm, pooled and the buffer was exchanged to PBS by dialysis.

6. SDS-PAGE and Western Blotting

Samples were separated on 12% polyacrylamide gels under reducing conditions and on 8% polyacrylamide gels under non-reducing conditions, followed by either staining with Coomassie Brilliant Blue or blotting onto a nitrocellulose membrane (GE Healthcare). mAb heavy and light chains were detected with anti-human IgG gamma chain-peroxidase conjugate (Sigma A8775) or anti-human lambda light chain-peroxidase conjugate (Sigma A5175) and visualized with a chemiluminescence detection kit (Bio-Rad).

7. Glycosylation Analysis of mAbs and gp120

The N-glycosylation profiles of mAbs (Asn²⁹⁷) and gp120^{ZM109} (Asn¹⁶⁰, Asn¹⁷³) were determined by LC-ESI-MS as published by Stadlmann et al. (Proteomics 8(2008):2858-2871) and Pabst et al. (Biol Chem 393(2012):719-730), respectively. Briefly, purified mAbs or gp120 were separated by reducing SDS-PAGE, stained with Coomassie Brilliant Blue and the relevant bands were excised from the gel. Upon S-alkylation with iodoacetamide and tryptic or tryptic/chymotryptic digestion (gp120^{ZM109} Asn¹⁷³), fragments were eluted from the gel with 50% acetonitrile and separated on a reversed phase column (150×0.32 mm BioBasic-18, Thermo Scientific) with a gradient of 1-80% acetonitrile. Glycopeptides were analysed with a Q-TOF Ultima Global mass spectrometer (Waters). Spectra were summed and deconvoluted for identification of glycoforms. Annotation of glycoforms was done according to the proglycan nomenclature (Stadlmann J. et al. (Proteomics 8(2008), 2858-2871).

8. Sulfation Analysis of PG9 and RSH

Tryptic peptides were prepared as above (see item 7), digested with AspN where appropriate and then separated using a Dionex Ultimate 3000 HPLC system using a Thermo BioBasic C18 separation column (5 μm particle size, 150×0.32 mm) with a gradient from 95% solvent A (65 mM ammonium formate) and 5% solvent B (acetonitrile) to 75% B in 50 min at a flow rate of 6 μL/min. Peptides were analysed on a maXis 4G ETD QTOF mass spectrometer (Bruker Daltonik) equipped with the standard ESI source in the positive ion, DDA mode (=switching to MSMS mode for eluting peaks). MS2 scans of dominant precursor peaks were acquired and manually analysed with DataAnalysis software version 4.0 (Bruker Daltonik).

9. Quantification of mAb Content and gp120 Binding by ELISA

For determination of PG9 and RSH content, wells were coated with 100 μl of 2 μg/ml anti-human gamma chain (Sigma-Aldrich 13391) in 0.1 N NaHCO₃ buffer (pH 9.6) overnight at 4° C. After washing with PBS containing 0.05% Tween-20 (PBST), samples and ^{CHO}PG9 standards (100 μl) appropriately diluted (1 - 100 ng/ml) in PBST containing 1% BSA were added, the plate was incubated for 60 min, washed and incubated for 60 min with 100 μl of a 1:20.000 dilution of anti-human lambda light chain-peroxidase conjugate (Sigma A5175). After washing with PBST, the wells were incubated for 20 min with 100 μl 3,3′,5,5′-tetramethylbenzidine (TMB) substrate solution (Sigma-Aldrich T0440). The reaction was then stopped with 100 μl 30% H₂SO₄ prior to spectrophotometry at 450 nm.

To test binding to gp120, the ELISA setup was adopted as follows: 1 μg/ml gp120^{ZM109} was coated, and the mAb sample concentrations ranged from 10 ng/ml to 4 μg/ml. As detection antibody a 1:2.000 dilution of anti-human IgG (Fcgamma specific)peroxidase conjugate (Invitrogen 62-8420) was used.

10. Biolayer Interferometry

PG9 was bound at 20 μg/ml to Dip and Read Protein A Biosensor sticks (fortéBio) and antigen binding kinetics were determined with gp120^{ZM109} solutions ranging from 50 μg/ml to 6.25 μg/ml (1:2 dilutions). Blanks were run without PG9 and/or without gp120^{ZM109}. All measurements were conducted at 30° C. Results were analysed with Octet Data Analysis Software 6.4 with single reference-well subtractions. The kinetic constants were computed for each curve separately assuming that dissociation does not reach the pre-association baseline. All estimates with a coefficient of determination (R²) above 0.85 were considered for calculation of the dissociation constant K_{d}.

11. Virus Neutralization Assays

Pseudotyped virions were generated as described previously (Gach J S, et al. PLoS One 8(2013):e72054). In brief, 5×10⁵ human embryonic kidney 293T cells (ATCC, # CRL-3216) were cotransfected with 4 μg of the HIV Env-deleted backbone plasmid pSG3ΔEnv and 2 μg of the respective Env complementation plasmid using polyethyleneimine (18 μg) as a transfection reagent. Cell culture supernatants were harvested 48 h after transfection, cleared by centrifugation at 4.000 g for 10 min, and then used for single-round infectivity assays as described elsewhere (Gach J S, et al. PLoS One 9(2014):e85371). Briefly, pseudotyped virus was added at a 1:1 volume ratio to serially diluted (1:3) mAbs (starting at 40 μg/ml) and incubated at 37° C. After 1 h TZM-bl reporter cells (NIH AIDS Reagent Program, # 8129) were added (1:1 by volume) at 1×10⁴ cells/well, supplemented with 10 μg/ml DEAE-dextran and then incubated for a further 48 h at 37° C. Next, the cells were washed, lysed, and developed with luciferase assay reagent according to the manufacturer's instructions (Promega). Relative light units were then measured using a microplate luminometer (BioTek, Synergy 2 luminescence microplate reader). All experiments were performed at least in duplicate. The extent of virus neutralization in the presence of antibody was determined as the 50% or 90% inhibitory concentration (IC₅₀, IC₉₀) as compared to samples treated without mAb.

12. Protein Quantification

The total protein content of gp120 and mAb samples was quantified with the BCA Protein Assay Kit (Pierce) using BSA as standard according to the protocol provided by the manufacturer.

### Example 1

### Natural ^{ΔXF}PG9 Binds gp120 Less Efficiently than ^{CHO}PG9

Previously the in planta production of PG9 in ΔXT/FT Nicotiana benthamiana plants that have been glycoengineered to remove the plant-typical N-glycan residues β1,2-xylose and core α1,3-fucose has been reported (Niemer M, et al. Biotech J 9(2014):493-500). Another recent study has revealed that changing three consecutive amino acids of the PG9 light chain into the corresponding PG16 residues (T^{L94}RR^{L95A} to R^{L94}SH^{L95A}) leads to improved antigen-binding characteristics and higher neutralization efficiency (Pancera M, et al. Nat Struct Mol Biol 20(2013):804-+). Therefore, a ^{ΔXF}PG9-R^{L94}SH^{L95A} variant termed ^{ΔXF}RSH was constructed. Using protein A affinity chromatography, ^{ΔXF}PG9 and ^{ΔXF}RSH could be purified in good yields from leaf extracts. When analyzed by SDS-PAGE under reducing conditions, the ^{ΔXF}PG9 and ^{ΔXF}RSH heavy and light chains showed the expected migration pattern, with the light chains displaying higher electrophoretic mobilities than their CHO-derived counterpart (^{CHO}PG₉) due to the removal of a functionally unnecessary N-glycosylation site. Under non-reducing conditions, ^{ΔXF}PG9 and ^{ΔXF}RSH yielded single major bands co-migrating with ^{CHO}PG9 (FIG. 2).

To investigate the antigen-binding properties of ^{ΔXF}PG9 in comparison to ^{CHO}PG9, suitable ligand was required. PG9 has been described to bind with high affinity to trimeric envelope glycoproteins of a wide variety of HIV isolates and also to gp120 monomers of selected HIV strains including ZM109. Therefore gp120^{ZM109} containing a C-terminal hexahistidine tag was expressed in FreeStyle 293 (FS293) cells and purified by metalchelate affinity chromatography to apparent homogeneity. SDSPAGE revealed a diffuse band as expected for a heavily glycosylated protein. N-glycosylation of two gp120 asparagines (Asn¹⁶⁰ and Asn¹⁷³) has been shown to be important for PG9 binding. Glycosylation analysis by mass spectrometry revealed mainly Man5 structures on either of these N-glycosylation sites. Importantly, PG9 is known to prefer such N-glycans on Asn¹⁶⁰ while tolerating them on Asn¹⁷³. Only minor amounts of other N-glycans were detected on either site, indicating that FS293-derived gp120^{ZM109} meets the prerequisites for a high-affinity PG9 ligand. When tested by ELISA, binding of ^{ΔXF}PG9 to gp120^{ZM109} was found to be considerably weaker than observed for ^{CHO}PG9 (Table A).

TABLE A
Sulfation enhances binding of PG9 and RSH to gp120/140.
Binding of PG9 and RSH antibodies to immobilized antigen
was measured by ELISA in triplicates (monomeric gp120^{ZM109})
or duplicates (trimeric gp140^{BG505.SOSIP.664}). Data are
presented as means ± SD.

EC₅₀ [ng/ml]

| mAb | gp120 | gp140 |
|---|---|---|
| ^{CHO}PG9 | 89 ± 3 | 290 ± 120 |
| ^{ΔXF}PG9 | 452 ± 72 | 4870 ± 1560 |
| ^{ΔXF}PG9_{Sulf} | 92 ± 16 | 490 ± 240 |
| ^{ΔXF}PG9_{SulfSia} | 101 ± 12 | 310 ± 50 |
| ^{ΔXF}RSH | 179 ± 85 | 2230 ± 170 |
| ^{ΔXF}RSH_{Sulf} | 82 ± 33 | 180 ± 10 |
| ^{ΔXF}RSH_{SulfSia} | 73 ± 22 | 180 ± 40 |

### Example 2

### Co-expression of TPST1 Enables the in Planta Production of Sulfated PG9

Sulfation of one or two tyrosine residues in the CDR H3 domain of PG9 and other V1/V2-directed bnAbs has been described to enhance antigen binding. However, a functional tyrosylprotein sulfotransferase is not contained in the N. benthamiana genome. Therefore, it is obvious that ^{ΔXF}PG9 was not properly sulfated. Analysis of CDR H3 peptides by mass spectrometry revealed a high degree (82%) of sulfation in the case of ^{CHO}PG9, whereas this post-translational modification was not detected for ^{ΔXF}PG9 (Table B).

TABLE B
Tyrosine sulfation of plant-produced PG9 and RSH. The
sulfation status of ^{CHO}PG9 is shown for comparison.

| | ^{CHO}PG9 | PG9 | PG9_{Sulf} | PG9_{SulfSia} | RSH | RSH_{Sulf} | RSH_{SulfSia} |
|---|---|---|---|---|---|---|---|
| 0S [%] | 18.5 | >98 | 49.2 | 42.9 | >98 | 52.7 | 48.9 |
| 1S [%] | 60.4 | <1 | 33.2 | 33.8 | <1 | 30.5 | 34.3 |
| 2S [%] | 21.1 | <1 | 17.6 | 23.3 | <1 | 16.8 | 16.8 |
| 1 + 2S [%] | 81.5 | <2 | 50.8 | 57.1 | <2 | 47.3 | 51.1 |

Therefore, ^{ΔXF}PG9 was co-expressed with human TPST1 (hsTPST1) in N. benthamiana. To mediate proper targeting to sub-Golgi compartments, three constructs carrying different cytoplasmic tail, transmembrane domain and stem (CTS) regions were tested. Expression of hsTPST1 in combination with its authentic CTS region (p^{Full}hsTPST1) led to 15-20% sulfated ^{ΔXF}PG9 (Table C). Interestingly, replacement of the natural hsTPST1 CTS region with the corresponding parts of glycosylation enzymes known to be targeted to the trans-region of the plant Golgi (p^{Fut11}hsTPST1 and p^{RST}hsTPST1) led to a substantially higher level of ^{ΔXF}PG9 sulfation, almost reaching the extent of tyrosine sulfation observed for ^{CHO}PG9. Using p^{RST}hsTPST1, up to 57% of ^{ΔXF}PG9 could be mono- or disulfated (Table B; Table C).

TABLE C
Tyrosine sulfation of PG9 and RSH. Relative amounts of
unsulfated (0S), singly (1S) and doubly (2S) sulfated PG9 and
RSH when coexpressed with different hsTPST1 constructs in
plants. The sulfation status of ^{CHO}PG9 is shown for comparison.
Data are presented as means ± SD of 2-9 analyses.

| mAb | TPST1 | OD₆₀₀ | 0S | 1S | 2S |
|---|---|---|---|---|---|
| ^{ΔXF}PG9_{Sulf} | p^{Full}hsTPST1 | 0.01 | 91.5 ± 7.0 | 8.2 ± 6.4 | 0.3 ± 0.7 |
| ^{ΔXF}PG9_{Sulf} | p^{Full}hsTPST1 | 0.05 | 83.4 ± 13.9 | 16 ± 13.1 | 0.6 ± 0.9 |
| ^{ΔXF}PG9_{Sulf} | p^{Full}hsTPST1 | 0.2 | 83.7 ± 8.8 | 15.6 ± 8.2 | 0.7 ± 0.9 |
| ^{ΔXF}PG9_{Sulf} | p^{Full}hsTPST1 | 0.8 | 82.8 ± 11.7 | 16.8 ± 11 | 0.4 ± 0.7 |
| ^{ΔXF}PG9_{Sulf} | p^{Fut11}hsTPST1 | 0.01 | 73.7 ± 5.8 | 17.8 ± 3.3 | 8.6 ± 2.6 |
| ^{ΔXF}PG9_{Sulf} | p^{Fut11}hsTPST1 | 0.05 | 57.4 ± 0.8 | 32.1 ± 2.3 | 10.6 ± 3.1 |
| ^{ΔXF}PG9_{Sulf} | p^{Fut11}hsTPST1 | 0.2 | 59.6 ± 5.8 | 28.1 ± 5.4 | 12.3 ± 5.4 |
| ^{ΔXF}PG9_{Sulf} | p^{Fut11}hsTPST1 | 0.8 | 58.7 ± 8.7 | 32.7 ± 10.8 | 8.6 ± 2.1 |
| ^{ΔXF}PG9_{Sulf} | p^{RST}hsTPST1 | 0.01 | 69.5 ± 8.9 | 20.6 ± 6.9 | 9.9 ± 2.3 |
| ^{ΔXF}PG9_{Sulf} | p^{RST}hsTPST1 | 0.05 | 53.9 ± 6.1 | 30.8 ± 2.3 | 15.3 ± 3.8 |
| ^{ΔXF}PG9_{Sulf} | p^{RST}hsTPST1 | 0.2 | 57.2 ± 10.4 | 29.3 ± 7.4 | 13.5 ± 5.1 |
| ^{ΔXF}PG9_{Sulf} | p^{RST}hsTPST1 | 0.8 | 53.7 ± 11.1 | 36.8 ± 11.8 | 9.5 ± 2.7 |
| ^{ΔXF}RSH_{Sulf} | p^{RST}hsTPST1 | 0.2 | 57.4 ± 7.6 | 29.6 ± 5.2 | 13.1 ± 3.6 |
| ^{CHO}PG9 | | | 18.5 ± 1.5 | 60.4 ± 3.9 | 21.1 ± 5.4 |

### Example 3

### Tyrosine Sulfation of PG9 Produced in Plants and CHO Cells Occurs at the Same Positions

The tryptic CDR H3 peptide used for analyzing the PG9 sulfation status by LC-ESI-MS (N^{100C}GYNYYDFYDGYYNYHYMDVWGK¹⁰⁵; SEQ ID No. 27) contains several tyrosine residues that are potential TPST targets. To narrow down which amino acids are sulfated in the case of plant-derived PG9, the tryptic peptide was further digested with AspN to give rise to the shorter peptide N^{100C}GYNYY^{100H} (SEQ ID No. 28), containing the tyrosines involved in gp120 binding, as well as D^{100I}FYDGYYNYHYM^{100T} (SEQ ID No. 29) and D¹⁰¹WGK¹⁰⁵. In plant-produced as well as CHO-derived PG9 and RSH, no sulfates were found on D^{100I}FYDGYYNYHYM^{100T} (SEQ ID No. XX) (FIG. 3), whereas N^{100C}GYNYY^{100H} (SEQ ID No. 28) was found to be singly and doubly sulfated to roughly the same extent as N^{100C}GYNYYDFYDGYYNYHYMDVWGK¹⁰⁵ (SEQ ID No. 27). This indicates that the sulfate groups are attached to Y^{100E}, Y^{100G} and/or Y^{100H} independent of the expression platform used for PG9 production. It has been shown previously by X-ray crystallography that Y^{100G} and Y^{100H} of mammalian cell-produced PG9 can be sulfated. This shows that human TPST1 also modifies the same tyrosine residues in planta.

### Example 4

### PG9 Carries Human-type N-glycans when Expressed in Glycoengineered Plants

Mass spectrometric N-glycan analysis of ^{ΔXF}PG9, ^{ΔXF}PG9_{Sulf}, ^{ΔXF}RSH and ^{ΔXF}RSH_{Sulf} revealed the presence of a single dominant N-glycan species, GnGn (G0). This glycoform accounted for roughly 45-50% of all N-glycan species. Upon coexpression of PG9 and RSH with mammalian genes necessary for terminal galactosylation and sialylation in planta resulting in the synthesis of ^{ΔXF}PG9_{SulfSia} and _{ΔXF}RSH_{SulfSia} the N-glycosylation profiles shifted to 30-40% galactosylated oligosaccharides and 6-12% sialylated glycans, with G0 reduced to 15-20%. Importantly, core α1,3-fucose and β1,2-xylose residues were barely detectable in all 6 plant-produced variants (below 5%). In the case of ^{CHO}PG9, the vast majority of Asn²⁹⁷ N-glycans contained α1,6-fucose (more than 95%) and the main N-glycan structure detected (70%) was G0F⁶ (GnGnF⁶). Roughly 20% of ^{CHO}PG9 was galactosylated and less than 1% sialylated. These results indicate that the N-glycan moieties of ^{ΔXF}PG_{SulfSia} and ^{ΔXF}RSH_{SulfSia} are largely devoid of the core fucose residues known to hamper mAb binding to Fc receptors while otherwise being reminiscent of those found on PG9 produced in mammalian cell factories.

### Example 5

### Antigen Binding by ^{ΔXF}PG9 is Enhanced by Tyrosine Sulfation

Binding of the different PG9 and RSH variants to monomeric gp120^{ZM109} or trimeric gp140^{BG505.SOSIP.664} was tested by ELISA (Table A), and EC₅₀ values were calculated. RSH showed up to 3-fold better binding to either antigen than PG9. Sulfation of plant-produced PG9 and RSH increased their affinities 10-16 times for trimeric gp140 and 2-5 times for monomeric gp120^{ZM109}. As expected, different glycoforms showed very similar EC₅₀ values. The avidity of the antigen-antibody interaction was also determined by biolayer interferometry (Table D).

TABLE D
Affinities of PG9 and RSH for gp120^{ZM109} as determined
by biolayer interferometry measurements. Data are presented
as means ± SD of 2 (^{ΔXF}RSH) or 4-6 individual
determinations. The binding of ^{ΔXF}PG9 to gp120^{ZM109} was
too weak for accurate determination of K_{d} under the
experimental conditions used.

| | K_{d} [nM] |
|---|---|
| CHO_{PG9} | 756 ± 365 |
| ^{ΔXF}PG9_{Sulf} | 525 ± 167 |
| ^{ΔXF}RSH_{Sulf} | 605 ± 239 |
| ^{ΔXF}RSH | 2510 ± 39 |
| ^{ΔXF}PG9 | >3000 |

^{ΔXF}PG9_{Sulf}, ^{ΔXF}RSH_{Sulf} and ^{CHO}PG9 showed roughly the same affinity to the antigen (K_{d} of 525, 605 and 756 nM, respectively), whereas unsulfated ^{ΔXF}RSH exhibited a roughly 4-fold lower affinity (K_{d} of 2.51 μM). The results obtained by biolayer interferometry confirmed those from the ELISA experiments, namely that RSH binds stronger to gp120/gp140 than wild-type PG9 and that tyrosine sulfation increases the affinity of both antibodies for either antigen. Furthermore, ^{ΔXF}PG⁹_{Sulf} and ^{ΔXF}PG9_{SulfSial} displayed essentially the same gp120/140-binding properties as ^{CHO}PG9, demonstrating the suitability of our plant-based expression platform to produce fully active versions of this bnAb.

### Example 6

### Increased Virus Neutralization by Sulfated PG9 and RSH Variants

Finally, the neutralization efficiencies of the antibodies on a panel of HIV clade B and clade C pseudoviruses were tested (Table E).

TABLE E
Neutralization efficiencies of PG9 and RSH against a panel of pseudoviruses.
IC₅₀ values (μg/ml) are indicated as >50 μg/ml; 10-50 μg/ml; 1-10 μg/ml; <1 μg/ml).

| | ^{CHO}PG9 | ^{ΔXF}PG9 | ^{ΔXF}PG9_{Sulf} | ^{ΔXF}PG9_{SulfSia} | ^{ΔXF}RSH | ^{ΔXF}RSH_{Sulf} | ^{ΔXF}RSH_{SulfSia} |
|---|---|---|---|---|---|---|---|
| JRFL | >50 | >50 | >50 | >50 | >50 | >50 | >50 |
| PVO | >50 | >50 | >50 | >50 | >50 | >50 | >50 |
| TRO.11 | >50 | >50 | >50 | >50 | >50 | >50 | >50 |
| ZM214M | >50 | >50 | >50 | >50 | >50 | >50 | >50 |
| YU2 | >50 | >50 | >50 | >50 | 42.47 | 34.75 | 26.08 |
| MN | >50 | >50 | >50 | >50 | 43.70 | 30.77 | 36.14 |
| ADA | 42.81 | >50 | 37.36 | 40.17 | 26.15 | 19.58 | 20.06 |
| DU422.1 | 10.93 | >50 | 14.28 | 6.60 | >50 | 9.72 | 8.45 |
| ZM109F | 0.78 | >50 | 1.66 | 1.31 | >50 | 1.46 | 1.38 |
| DU156.12 | 0.35 | >50 | 1.20 | 0.90 | 33.85 | 0.56 | 0.56 |
| CAP45 | <0.02 | 0.65 | 0.03 | <0.02 | 0.25 | <0.02 | <0.02 |
| JRCSF | <0.02 | 0.19 | <0.02 | 0.03 | 0.06 | <0.02 | <0.02 |

The viruses included well-neutralized isolates as well as some resistant to PG9 or RSH produced in mammalian cells. As expected, a number of pseudoviruses was not neutralized under the tested conditions (JRFL, ZM214M, PVO, TRO.11), whereas others were neutralized at intermediate (ADA, YU2, MN) to good efficiency (DU156.12, DU422.1, ZM109) and some were neutralized very efficiently (JRCSF, CAP45). Interestingly, the various PG9 variants displayed pronounced differences with respect to their neutralization efficiencies. In accordance with the results of the antigen-binding assays, tyrosine sulfation strongly enhanced neutralization of highly sensitive isolates (50-fold and more; e.g. JRCSF, DU156.12, ZM109, CAP45, DU422.1), whereas only a modest improvement was observed for more resistant strains (1.3-1.5 fold; ADA, YU2 and MN). These data provide unprecedented evidence for the pivotal role of CDR H3 sulfotyrosines in effective HIV neutralization by PG9 as previously proposed based on the tertiary structure of the PG9/gp120 complex. In general, the varying sensitivities of the tested HIV strains to PG9 and RSH were in good agreement with the presence or absence of PG9-interacting residues in their gp120 V2 sequences. Furthermore, the observed differences in neutralization efficiency between PG9 and RSH were comparable to those found in antigen-binding assays. Importantly, glycoengineering of plant-derived PG9 and RSH did not affect virus neutralization, thus demonstrating that fine tuning of Asn²⁹⁷ N-glycosylation does not compromise the anti-viral potency of these bnAbs.

### Conclusion

The examples provided herein aimed at the establishment of sulfoengineering in plants in order to increase the in vivo efficiency of two HIV-specific mAbs, PG9 and RSH. Although some plants have a tyrosylprotein sulfotransferase and can sulfate phytohormones, PG9 expressed in N. benthamiana did not contain detectable amounts of sulfated peptides, indicating that mammalian-type sulfation does not occur naturally in N. benthamiana leaves. In humans, sulfation of suitable tyrosine residues is carried out by the two tyrosylprotein sulfotransferases hsTPST1 and hsTPST2, and overexpression of hsTPST1 and hsTPST2 has been shown to increase sulfation of recombinantly produced proteins in CHO and HEK293T cells. However, expression of full-length hsTPST1 did not yield high levels of sulfation in N. benthamiana. Replacing the authentic CTS sequence of hsTPST1 with a plant CTS region, for instance, drastically increased the sulfation efficiency and led to mAbs with an increased neutralization efficacy.

The crystal structure of PG9 in complex with its antigen has revealed that Y^{100H} and Y^{100G}of the PG9 (and RSH) heavy chain can be sulfated and that their sulfation increases antigen binding. By mass spectrometry, the sulfation sites of ^{CHO}PG9 and plant-produced PG9/RSH could be mapped to a short peptide containing 3 tyrosine residues (Y^{100E}, Y^{100H} and Y^{100G}). Sulfation of plant-produced PG9 and RSH enhanced antigen binding and virus neutralization, indicating that also in plants Y^{100H} and Y^{100G} are the sulfated residues. While the impact of tyrosine sulfation on neutralization efficiency was so far only assessed for singly and doubly sulfated PG9, sulfated and unmodified PG9/RSH were compared and a far more pronounced difference in anti-viral potency was observed. These results show that singly sulfated PG9 binds and neutralizes HIV better than non-sulfated antibody, and that the doubly sulfated mAb has an even higher efficacy.

1. A nucleic acid molecule encoding
a) a modified tyrosylprotein sulfotransferase of a wildtype tyrosylprotein sulfotransferase, wherein the cytoplasmic transmembrane stem (CTS) region of the wild-type tyrosylprotein sulfotransferase is replaced by a heterologous CTS region, or
b) a fusion protein comprising a catalytically active fragment of a tyrosylprotein sulfotransferase fused to a heterologous CTS region.

2. Nucleic acid molecule according to claim 1, wherein the heterologous CTS region is a plant or animal CTS region.

3. Nucleic acid molecule according to claim 1 or 2, wherein the heterologous CTS region is a CTS region of a glycosyltransferase or a tyrosylprotein sulfotransferase.

4. Nucleic acid molecule according to claim 3, wherein the glycosyltransferase is selected from the group consisting of a fucosyltransferase, preferably an α1,3-fucosyltransferase, more preferably an α1,3-Fucosyltransferase 11, and a sialytransferase, preferably an α2,6-sialytransferase.

5. Nucleic acid molecule according to any one of claims 1 to 4, wherein the tyrosylprotein sulfotransferase is a plant or animal tyrosylprotein sulfotransferase.

6. Nucleic acid molecule according to any one of claims 1 to 5, wherein the heterologous CTS region of said fusion protein is N- or C-terminally, preferably N-terminally, fused to the tyrosylprotein sulfotransferase or the catalytically active fragment thereof.

7. Polypeptide encoded by a nucleic acid molecule according to any one of claims 1 to 6.

8. A vector comprising a nucleic acid molecule according to any one of claims 1 to 6.

9. A plant or plant cell capable of transferring a sulfate moiety to a tyrosine residue of a polypeptide heterologously produced in said plant or plant cell comprising a nucleic acid molecule according to any one of claims 1 to 6 or a vector according to claim 8.

10. Plant or plant cell according to claim 9, wherein said transgenic plant or plant cell comprises further a nucleic acid molecule encoding a heterologous polypeptide operably linked to a promoter region.

11. Plant or plant cell according to claim 10, wherein the heterologous polypeptide of animal origin is a mammalian, more preferably human, polypeptide.

12. Plant or plant cell according to claim 11, wherein the heterologous animal polypeptide is an antibody.

13. Plant or plant cell according to claim 12, wherein the antibody is an antibody binding to an HIV surface protein.

14. Plant or plant cell according to claim 12 or 13, wherein the antibody is an antibody selected from the group consisting of PG9, PG16, PGT141-145, 47e, 412d, Sb1, C12, E51, CM51 and a variant thereof.

15. Plant or plant cell according to claim 14, wherein the antibody variant is a PG9 antibody comprising modifications at R^{L94}SH^{L95A}.

16. A method of recombinantly producing a polypeptide of animal origin carrying an animal-type sulfation comprising the step of cultivating a plant or plant cell according to any one of claims 9 to 15.


SEQUENCE LISTING (35 sequences)

SEQ ID NO: 1 - protein, 370 aa, Homo sapiens
MVGKLKQNLLLACLVISSVTVFYLGQHAMECHHRIEERSQPVKLESTRTTVRTGLDLKANKTFAYHKDMPLIFIGGVPRSGTTLMRAMLDAHPDIRCGEETRVIPRILALKQMWSRSSKEKIRLDEAGVTDEVLDSAMQAFLLEIIVKHGEPAPYLCNKDPFALKSLTYLSRLFPNAKFLLMVRDGRASVHSMISRKVTIAGFDLNSYRDCLTKWNRAIETMYNQCMEVGYKKCMLVHYEQLVLHPERWMRTLLKFLQIPWNHSVLHHEEMIGKAGGVSLSKVERSTDQVIKPVNVGALSKWVGKIPPDVLQDMAVIAPMLAKLGYDPYANPPNYGKPDPKIIENTRRVYKGEFQLPDFLKEKPQTEQVE

SEQ ID NO: 2 - DNA, 1113 bp, Homo sapiens
atggttggaaagctgaagcagaacttactattggcatgtctggtgattagttctgtgactgtgttttacctgggccagcatgccatggaatgccatcaccggatagaggaacgtagccagccagtcaaattggagagcacaaggaccactgtgagaactggcctggacctcaaagccaacaaaacctttgcctatcacaaagatatgcctttaatatttattggaggtgtgcctcggagtggaaccacactcatgagggccatgctggacgcacatcctgacattcgctgtggagaggaaaccagggtcattccccgaatcctggccctgaagcagatgtggtcacggtcaagtaaagagaagatccgcctggatgaggctggtgttactgatgaagtgctggattctgccatgcaagccttcttactagaaattatcgttaagcatggggagccagccccttatttatgtaataaagatccttttgccctgaaatctttaacttacctttctaggttattccccaatgccaaatttctcctgatggtccgagatggccgggcatcagtacattcaatgatttctcgaaaagttactatagctggatttgatctgaacagctatagggactgtttgacaaagtggaatcgtgctatagagaccatgtataaccagtgtatggaggttggttataaaaagtgcatgttggttcactatgaacaacttgtcttacatcctgaacggtggatgagaacactcttaaagttcctccagattccatggaaccactcagtattgcaccatgaagagatgattgggaaagctgggggagtgtctctgtcaaaagtggagagatctacagaccaagtaatcaagccagtcaatgtaggagctctatcaaaatgggttgggaagataccgccagatgttttacaagacatggcagtgattgctcctatgcttgccaagcttggatatgacccatatgccaacccacctaactacggaaaacctgatcccaaaattattgaaaacactcgaagggtctataagggagaattccaactacctgactttcttaaagaaaaaccacagactgagcaagtggagtag

SEQ ID NO: 3 - protein, 39 aa, Artificial Sequence
  CTS region of SEQ ID No. 1
MVGKLKQNLLLACLVISSVTVFYLGQHAMECHHRIEERS

SEQ ID NO: 4 - DNA, 117 bp, Artificial Sequence
  Nucleic acid molecule encoding a CTS region
atggttggaaagctgaagcagaacttactattggcatgtctggtgattagttctgtgactgtgttttacctgggccagcatgccatggaatgccatcaccggatagaggaacgtagc

SEQ ID NO: 5 - protein, 66 aa, Artificial Sequence
  CTS region
MGVFSNLRGPKIGLTHEELPVVANGSTSSSSSPSSFKRKVSTFLPICVALVVIIEIGFLCRLDNAS

SEQ ID NO: 6 - DNA, 198 bp, Artificial Sequence
  Nucleic acid sequence encoding a CTS region
atgggtgttttctccaatcttcgaggtcctaaaattggattgacccatgaagaattgcctgtagtagccaatggctctacttcttcttcttcgtctccttcctctttcaagcgtaaagtctcgacctttttgccaatctgcgtggctcttgtcgtcattatcgagatcgggttcctctgtcggctcgataacgcttct

SEQ ID NO: 7 - protein, 52 aa, Artificial Sequence
  CTS region
MIHTNLKKKFSLFILVFLLFAVICVWKKGSDYEALTLQAKEFQMPKSQEKVA

SEQ ID NO: 8 - DNA, 156 bp, Artificial Sequence
  Nucleic acid sequence encoding a CTS region
atgattcataccaacttgaagaaaaagttcagcctcttcatcctggtctttctcctgttcgcagtcatctgtgtttggaagaaagggagcgactatgaggcccttacactgcaagccaaggaattccagatgcccaagagccaggagaaagtggcc

SEQ ID NO: 9 - DNA, 1195 bp, Artificial Sequence
  Nucleic acid encoding a human tyrosylprotein sulfotransferase whose wild-type CTS region at its N-terminus has been replaced by a CTS region of alpha-1,3-Fucosyltransferase 11 of Arabidopsis thaliana
  misc_feature (199)..(199): n=1 to 10 nucleic acid resiudes
atgggtgttttctccaatcttcgaggtcctaaaattggattgacccatgaagaattgcctgtagtagccaatggctctacttcttcttcttcgtctccttcctctttcaagcgtaaagtctcgacctttttgccaatctgcgtggctcttgtcgtcattatcgagatcgggttcctctgtcggctcgataacgcttctncagccagtcaaattggagagcacaaggaccactgtgagaactggcctggacctcaaagccaacaaaacctttgcctatcacaaagatatgcctttaatatttattggaggtgtgcctcggagtggaaccacactcatgagggccatgctggacgcacatcctgacattcgctgtggagaggaaaccagggtcattccccgaatcctggccctgaagcagatgtggtcacggtcaagtaaagagaagatccgcctggatgaggctggtgttactgatgaagtgctggattctgccatgcaagccttcttactagaaattatcgttaagcatggggagccagccccttatttatgtaataaagatccttttgccctgaaatctttaacttacctttctaggttattccccaatgccaaatttctcctgatggtccgagatggccgggcatcagtacattcaatgatttctcgaaaagttactatagctggatttgatctgaacagctatagggactgtttgacaaagtggaatcgtgctatagagaccatgtataaccagtgtatggaggttggttataaaaagtgcatgttggttcactatgaacaacttgtcttacatcctgaacggtggatgagaacactcttaaagttcctccagattccatggaaccactcagtattgcaccatgaagagatgattgggaaagctgggggagtgtctctgtcaaaagtggagagatctacagaccaagtaatcaagccagtcaatgtaggagctctatcaaaatgggttgggaagataccgccagatgttttacaagacatggcagtgattgctcctatgcttgccaagcttggatatgacccatatgccaacccacctaactacggaaaacctgatcccaaaattattgaaaacactcgaagggtctataagggagaattccaactacctgactttcttaaagaaaaaccacagactgagcaagtggagtag

SEQ ID NO: 10 - DNA, 1153 bp, Artificial Sequence
  Nucleic acid sequence encoding a human tyrosylprotein sulfotransferase whose wild-type CTS region at its N-terminus has been replaced by a CTS region of an alpha-2,6-sialytransferase of Rattus norvegicus
  misc_feature (157)..(157): n=1 to 10 nucleic acid resiudes
atgattcataccaacttgaagaaaaagttcagcctcttcatcctggtctttctcctgttcgcagtcatctgtgtttggaagaaagggagcgactatgaggcccttacactgcaagccaaggaattccagatgcccaagagccaggagaaagtggccncagccagtcaaattggagagcacaaggaccactgtgagaactggcctggacctcaaagccaacaaaacctttgcctatcacaaagatatgcctttaatatttattggaggtgtgcctcggagtggaaccacactcatgagggccatgctggacgcacatcctgacattcgctgtggagaggaaaccagggtcattccccgaatcctggccctgaagcagatgtggtcacggtcaagtaaagagaagatccgcctggatgaggctggtgttactgatgaagtgctggattctgccatgcaagccttcttactagaaattatcgttaagcatggggagccagccccttatttatgtaataaagatccttttgccctgaaatctttaacttacctttctaggttattccccaatgccaaatttctcctgatggtccgagatggccgggcatcagtacattcaatgatttctcgaaaagttactatagctggatttgatctgaacagctatagggactgtttgacaaagtggaatcgtgctatagagaccatgtataaccagtgtatggaggttggttataaaaagtgcatgttggttcactatgaacaacttgtcttacatcctgaacggtggatgagaacactcttaaagttcctccagattccatggaaccactcagtattgcaccatgaagagatgattgggaaagctgggggagtgtctctgtcaaaagtggagagatctacagaccaagtaatcaagccagtcaatgtaggagctctatcaaaatgggttgggaagataccgccagatgttttacaagacatggcagtgattgctcctatgcttgccaagcttggatatgacccatatgccaacccacctaactacggaaaacctgatcccaaaattattgaaaacactcgaagggtctataagggagaattccaactacctgactttcttaaagaaaaaccacagactgagcaagtggagtag

SEQ ID NO: 11 - protein, 398 aa, Artificial Sequence
  Human tyrosylprotein sulfotransferase whose wild-type CTS region at its N-terminus has been replaced by a CTS region of an alpha-1,3-Fucosyltransferase 11
  MISC_FEATURE (67)..(67): Xaa=1 to 5 amino acid residues
MGVFSNLRGPKIGLTHEELPVVANGSTSSSSSPSSFKRKVSTFLPICVALVVIIEIGFLCRLDNASXQPVKLESTRTTVRTGLDLKANKTFAYHKDMPLIFIGGVPRSGTTLMRAMLDAHPDIRCGEETRVIPRILALKQMWSRSSKEKIRLDEAGVTDEVLDSAMQAFLLEIIVKHGEPAPYLCNKDPFALKSLTYLSRLFPNAKFLLMVRDGRASVHSMISRKVTIAGFDLNSYRDCLTKWNRAIETMYNQCMEVGYKKCMLVHYEQLVLHPERWMRTLLKFLQIPWNHSVLHHEEMIGKAGGVSLSKVERSTDQVIKPVNVGALSKWVGKIPPDVLQDMAVIAPMLAKLGYDPYANPPNYGKPDPKIIENTRRVYKGEFQLPDFLKEKPQTEQVE

SEQ ID NO: 12 - protein, 384 aa, Artificial Sequence
  Human tyrosylprotein sulfotransferase whose wild-type CTS region at its N-terminus has been replaced by a CTS region of an alpha-2,6-sialytransferase of Rattus norvegicus
  MISC_FEATURE (53)..(53): Xaa=1 to 5 amino acid residues
MIHTNLKKKFSLFILVFLLFAVICVWKKGSDYEALTLQAKEFQMPKSQEKVAXQPVKLESTRTTVRTGLDLKANKTFAYHKDMPLIFIGGVPRSGTTLMRAMLDAHPDIRCGEETRVIPRILALKQMWSRSSKEKIRLDEAGVTDEVLDSAMQAFLLEIIVKHGEPAPYLCNKDPFALKSLTYLSRLFPNAKFLLMVRDGRASVHSMISRKVTIAGFDLNSYRDCLTKWNRAIETMYNQCMEVGYKKCMLVHYEQLVLHPERWMRTLLKFLQIPWNHSVLHHEEMIGKAGGVSLSKVERSTDQVIKPVNVGALSKWVGKIPPDVLQDMAVIAPMLAKLGYDPYANPPNYGKPDPKIIENTRRVYKGEFQLPDFLKEKPQTEQVE

SEQ ID NO: 13 - protein, 377 aa, Homo sapiens
MRLSVRRVLLAAGCALVLVLAVQLGQQVLECRAVLAGLRSPRGAMRPEQEELVMVGTNHVEYRYGKAMPLIFVGGVPRSGTTLMRAMLDAHPEVRCGEETRIIPRVLAMRQAWSKSGREKLRLDEAGVTDEVLDAAMQAFILEVIAKHGEPARVLCNKDPFTLKSSVYLSRLFPNSKFLLMVRDGRASVHSMITRKVTIAGFDLSSYRDCLTKWNKAIEVMYAQCMEVGKEKCLPVYYEQLVLHPRRSLKLILDFLGIAWSDAVLHHEDLIGKPGGVSLSKIERSTDQVIKPVNLEALSKWTGHIPGDVVRDMAQIAPMLAQLGYDPYANPPNYGNPDPFVINNTQRVLKGDYKTPANLKGYFQVNQNSTSSHLGSS

SEQ ID NO: 14 - DNA, 1189 bp, Homo sapiens
atgcgcctgtcggtgcggagggtgctgctggcagccggctgcgccctggtcctggtgctggcggttcagctgggacagcaggtgctagagtgccgggcggtgctggcgggcctgcggagcccccggggggccatgcggcctgagcaggaggagctggtgatggtgggcaccaaccacgtggaataccgctatggcaaggccatgccgctcatcttcgtgggtggcgtgcctcgcagtggcaccacgttgatgcgcgccatgctggacgcccaccccgaggtgcgctgcggcgaggagacccgcatcatcccgcgcgtgctggccatgcgccaggcctggtccaagtctggccgtgagaagctgcggctggatgaggcgggggtgacggatgaggtgctggacgccgccatgcaggccttcatcctggaggtgattgccaagcacggagagccggcccgcgtgctctgcaacaaggacccatttacgctcaagtcctcggtctacctgtcgcgcctgttccccaactccaagttcctgctgatggtgcgggacggccgggcctccgtgcactccatgatcacgcgcaaagtcaccattgcgggctttgacctcagcagctaccgtgactgcctcaccaagtggaacaaggccatcgaggtgatgtacgcccagtgcatggaggtaggcaaggagaagtgcttgcctgtgtactacgagcagctggtgctgcaccccaggcgctcactcaagctcatcctcgacttcctcggcatcgcctggagcgacgctgtcctccaccatgaagacctcattggcaagcccggtggtgtctccctgtccaagatcgagcggtccacggaccaggtcatcaagcctgttaacctggaagcgctctccaagtggactggccacatccctggggatgtggtgcgggacatggcccagatcgcccccatgctggctcagctcggctatgacccttatgcaaacccccccaactatggcaaccctgaccccttcgtcatcaacaacacacagcgggtcttgaaaggggactataaaacaccagccaatctgaaaggatattttcaggtgaaccagaacagcacctcctcccacttaggaagctcgtgatttccagatctccgcaaatgacttcattgccaagaagagaagaaaatgcatttaa

SEQ ID NO: 15 - protein, 370 aa, Mus musculus
MVGKLKQNLLLACLVISSVTVFYLGQHAMECHHRIEERSQPARLENPKATVRAGLDIKANKTFTYHKDMPLIFIGGVPRSGTTLMRAMLDAHPDIRCGEETRVIPRILALKQMWSRSSKEKIRLDEAGVTDEVLDSAMQAFLLEVIVKHGEPAPYLCNKDPFALKSLTYLARLFPNAKFLLMVRDGRASVHSMISRKVTIAGFDLNSYRDCLTKWNRAIETMYNQCMEVGYKKCMLVHYEQLVLHPERWMRTLLKFLHIPWNHSVLHHEEMIGKAGGVSLSKVERSTDQVIKPVNVGALSKWVGKIPPDVLQDMAVIAPMLAKLGYDPYANPPNYGKPDPKILENTRRVYKGEFQLPDFLKEKPQTEQVE

SEQ ID NO: 16 - protein, 390 aa, Mus musculus
MRRAPWLGLRPWLGMRLSVRKVLLAAGCALALVLAVQLGQQVLECRAVLGGTRNPRRMRPEQEELVMLGADHVEYRYGKAMPLIFVGGVPRSGTTLMRAMLDAHPEVRCGEETRIIPRVLAMRQAWTKSGREKLRLDEAGVTDEVLDAAMQAFILEVIAKHGEPARVLCNKDPFTLKSSVYLARLFPNSKFLLMVRDGRASVHSMITRKVTIAGFDLSSYRDCLTKWNKAIEVMYAQCMEVGRDKCLPVYYEQLVLHPRRSLKRILDFLGIAWSDTVLHHEDLIGKPGGVSLSKIERSTDQVIKPVNLEALSKWTGHIPRDVVRDMAQIAPMLARLGYDPYANPPNYGNPDPIVINNTHRVLKGDYKTPANLKGYFQVNQNSTSPHLGSS

SEQ ID NO: 17 - DNA, 1113 bp, Mus musculus
atggttgggaagctgaagcagaacttactcttggcgtgtctggtgattagttctgtgaccgtgttttacctgggccagcatgccatggagtgccatcaccgaatagaggaacgtagccagccagcccgactggagaaccccaaggcgactgtgcgagctggcctcgacatcaaagccaacaaaacattcacctatcacaaagatatgcctttaatattcatcgggggtgtgcctcggagcggcaccacactcatgagggctatgctggacgcacatcctgacatccgctgtggagaggaaaccagggtcatccctcgaatcctggccctgaagcagatgtggtcccggtccagtaaagagaagatccgcttggatgaggcgggtgtcacagatgaagtgctagattctgccatgcaagccttccttctggaggtcattgttaaacatggggagccggcaccttatttatgtaacaaagatccgtttgccctgaaatccttgacttaccttgctaggttatttcccaatgccaaatttctcctgatggtccgagatggccgggcgtcagtacattcaatgatttctcggaaagttactatagctggctttgacctgaacagctaccgggactgtctgaccaagtggaaccgggccatagaaaccatgtacaaccagtgtatggaagttggttataagaaatgcatgttggttcactatgaacagctcgtcttacaccctgaacggtggatgagaacgctcttaaagttcctccatattccatggaaccattccgttttgcaccatgaagaaatgatcgggaaagctgggggagtttctctgtcaaaggtggaaagatcaacagaccaagtcatcaaacccgtcaacgtgggggcgctatcgaagtgggttgggaagatacccccggacgtcttacaagacatggccgtgattgcacccatgctcgccaagcttggatatgacccatacgccaatcctcctaactacggaaaacctgaccccaagatccttgaaaacaccaggagggtctataaaggagaatttcagctccctgactttctgaaagaaaaaccccagacggagcaagtggagtaa

SEQ ID NO: 18 - DNA, 1173 bp, Mus musculus
atgaggcgggccccctggctgggcctgcgaccctggctgggcatgcgcctgtcggtgcgtaaggtgctgctggccgccggctgtgctctggccctggtgctcgctgtgcagcttgggcagcaagtactggagtgccgggcggtgctcgggggcacacggaacccacggaggatgcggccggagcaggaggaactggtgatgctcggcgccgaccacgtggagtaccgctatggcaaggccatgccactcatctttgtgggcggcgtgccacgcagtggcaccacgctcatgcgcgccatgttggacgcacacccagaggtgcgctgtggggaggagacgcgcatcatccctcgtgtgctggccatgcggcaggcctggaccaagtctggccgtgagaagctgcggctggacgaggcaggtgtgacggatgaggtgctggacgcggccatgcaggccttcattctggaggtgatcgccaagcacggcgaaccagcccgcgtgctgtgtaacaaggaccccttcacactcaagtcatccgtctacctggcacgcctgttccccaactccaaattcctgctaatggtgcgtgacggccgggcgtccgtgcactccatgatcacgcgcaaggtcaccatcgcgggctttgacctcagcagctaccgagactgcctcaccaagtggaacaaggccatcgaggtgatgtacgcacagtgcatggaggtgggcagggacaagtgcctgcccgtgtactatgagcagttggtgctgcacccccggcgctcactcaaacgcatcctggacttcctgggcatcgcctggagtgacacagtcctgcaccacgaggacctcattggcaagcctgggggcgtctccttgtccaagatcgagcggtccacggaccaggtcatcaaaccggtgaacttggaagctctctccaagtggacgggccacatccctagagacgtggtgagggatatggcccagattgcccccatgctggcccggcttggctatgacccgtatgcgaatccacccaactatgggaaccccgaccccattgtcatcaacaacacacaccgggtcttgaaaggagactataaaacgccagccaatctgaaaggatattttcaggtgaaccagaacagcacctccccacacctaggaagttcgtga

SEQ ID NO: 19 - protein, 380 aa, Caenorhabditis elegans
MRKNRELLLVLFLVVFILFYFITARTADDPYYSNHREKFNGAAADDGDESLPFHQLTSVRSDDGYNRTSPFIFIGGVPRSGTTLMRAMLDAHPEVRCGEETRVIPRILNLRSQWKKSEKEWNRLQQAGVTGEVINNAISSFIMEIMVGHGDRAPRLCNKDPFTMKSAVYLKELFPNAKYLLMIRDGRATVNSIISRKVTITGFDLNDFRQCMTKWNAAIQIMVDQCESVGEKNCLKVYYEQLVLHPEAQMRRITEFLDIPWDDKVLHHEQLIGKDISLSNVERSSDQVVKPVNLDALIKWVGTIPEDVVADMDSVAPMLRRLGYDPNANPPNYGKPDELVAKKTEDVHKNGAEWYKKAVQVVNDPGRVDKPIVDNEVSKL

SEQ ID NO: 20 - protein, 259 aa, Caenorhabditis elegans
MRAILDAHPDVRCGGETMLLPSFLTWQAGWRNDWVNNSGITQEVFDDAVSAFITEIVAKHSELAPRLCNKDPYTALWLPTIRRLYPNAKFILMIRDARAVVHSMIERKVPVAGYNTSDEISMFVQWNQELRKMTFQCNNAPGQCIKVYYERLIQKPAEEILRITNFLDLPFSQQMLRHQDLIGDEVDLNDQEFSASQVKNSINTKALTSWFDCFSEETLRKLDDVAPFLGILGYDTSISKPDYSTFADDDFYQFKNFYS

SEQ ID NO: 21 - DNA, 1143 bp, Caenorhabditis elegans
atgagaaaaaatcgagagttgctactcgtcctcttcctcgtcgtttttatactattctattttattactgcgagaactgcagacgacccgtactacagtaaccatcgggagaaattcaatggtgccgccgccgacgacggcgacgagtcgttaccttttcatcaattaacgtcagtacgaagtgatgatggatacaatagaacgtctcctttcatattcataggtggtgttcctcgctccggtacaactctgatgcgtgcgatgcttgacgctcatccagaagtcagatgtggtgaggagacacgtgtcattccacgcatcctgaatctacggtcacaatggaaaaagtcggaaaaggagtggaatcgactgcagcaggctggagtgacgggtgaagtgattaacaatgcgatcagctcgtttatcatggagataatggttggccacggagatcgggctcctcgtctctgcaacaaggatccattcacaatgaaatcagccgtctacctaaaagaactcttcccaaatgccaaatatcttctaatgatccgtgatggacgggccaccgtgaatagtataatctcacgaaaagtcacaattaccggattcgatttgaacgatttccgtcaatgcatgacgaaatggaatgcggcaattcaaataatggtagatcagtgtgaatcggttggagagaaaaattgtttgaaagtgtattatgagcagctggtgctacatccggaagcacaaatgcggcgaattacagagtttttggatattccgtgggatgataaagtgctgcaccatgagcagcttattggaaaagatatttctttatcgaatgtggaacggagctcggatcaagtcgttaaaccggttaatcttgatgctcttatcaaatgggttggaacgattcctgaggatgttgttgctgatatggattcggttgcgccgatgttaaggagattaggatatgatccgaatgcaaatccaccaaactatggaaaacccgacgaactagtcgcgaaaaaaacggaagatgttcataaaaatggagccgaatggtacaagaaagcagttcaagtggtcaacgatcccggccgcgtcgataaaccaattgttgataatgaagtatcgaaattatag

SEQ ID NO: 22 - DNA, 780 bp, Caenorhabditis elegans
atgagagctattctagatgcacatccggatgttcgatgtggcggtgaaaccatgctgcttccaagtttccttacatggcaagcaggctggcggaatgattgggtcaataattcaggaattactcaggaagtatttgacgacgctgtttcagcattcatcactgagatagtcgcgaagcacagtgaactagcacctcgtctgtgcaacaaggatccatacaccgcattgtggcttccgactattcgccgactgtacccgaatgcaaagtttattctgatgattcgagatgctcgtgccgtagttcattcaatgatagaaagaaaagtaccagttgctgggtataatacgtctgatgaaatttcaatgtttgttcagtggaatcaggagcttcgaaaaatgacttttcaatgcaataatgcgccagggcaatgcataaaagtatattatgaacgactgattcaaaaacctgcggaagaaatcctacgtatcaccaacttcctggatctgccattttcccagcaaatgctaagacatcaagatttaattggagacgaagttgatttaaacgatcaagaattctctgcatcacaagttaaaaactcgataaacactaaagccttaacctcgtggtttgattgttttagtgaagaaactctacgaaaacttgatgacgtggcaccttttttgggaattcttggatacgatacgtcgatttcaaaacccgattattccacatttgcggatgacgatttttaccaatttaaaaatttttattcttaa

SEQ ID NO: 23 - protein, 499 aa, Drosophila melanogaster
MRLPYRNKKVTLWVLFGIIVITMFLFKFTELRPTCLFKVDAANELSSQMVRVEKYLTDDNQRVYSYNREMPLIFIGGVPRSGTTLMRAMLDAHPDVRCGQETRVIPRILQLRSHWLKSEKESLRLQEAGITKEVMNSAIAQFCLEIIAKHGEPAPRLCNKDPLTLKMGSYVIELFPNAKFLFMVRDGRATVHSIISRKVTITGFDLSSYRQCMQKWNHAIEVMHEQCRDIGKDRCMMVYYEQLVLHPEEWMRKILKFLDVPWNDAVLHHEEFINKPNGVPLSKVERSSDQVIKPVNLEAMSKWVGQIPGDVVRDMADIAPMLSVLGYDPYANPPDYGKPDAWVQDNTSKLKANRMLWESKAKQVLQMSSSEDDNTNTIINNSNNKDNNNNQYTINKIIPEQHSRQRQHVQQQHLQQQQQQHLQQQQHQRQQQQQQREEESESEREAEPDREQQLLHQKPKDVITIKQLPLAGSNNNNINNNINNNNNNNNIMEDPMADT

SEQ ID NO: 24 - DNA, 1500 bp, Drosophila melanogaster
atgcgactgccatatcgaaataagaaggtcaccctgtgggtgctcttcggcatcatcgtcatcaccatgttcctattcaaattcaccgaactgcggcccacatgcctcttcaaggtggacgccgccaacgagctctcctcccaaatggttcgcgttgagaaatacctcacagatgacaatcaacgcgtttattcatacaaccgtgagatgccattaatattcataggcggcgtgccgagatctgggacgactttgatgcgcgccatgctggatgcccatcccgatgtgcgctgcgggcaggaaacccgtgtcattccgcgcatcctgcagctgcgctcgcactggctgaagtccgagaaggagtcgctccgcctgcaggaggccggcatcaccaaagaggtcatgaacagtgccatcgcgcagttctgtctggaaatcatcgccaaacacggcgagccggcgccgcgcttatgcaacaaggatccgctgacgctgaaaatgggctcctatgtcatcgagctatttccgaacgctaaattcctattcatggtgcgcgacggccgggcgacagttcattcgattatatcgcgcaaggtgacaatcaccggcttcgatttgagcagctaccggcagtgcatgcagaagtggaaccacgccatcgaggtgatgcacgagcagtgccgggacatcggcaaggaccgctgcatgatggtttactatgagcagctggtactgcatcccgaggagtggatgcgaaagatactgaaattcctggacgtgccatggaacgatgcggtgctgcaccacgaggagttcataaataaaccgaacggtgtgcctctgtccaaggtggaacgttcgtcggaccaggttatcaagccggttaatctggaggcgatgtccaaatgggttggccaaatacccggcgacgtggtgcgcgacatggccgacatagcgcccatgctgtccgtgctcggctacgatccgtacgcgaatccgccggactatggtaagccagatgcatgggtgcaggacaacacgtcgaagttaaaggccaatcgaatgctgtgggagagtaaggcgaagcaagtgctgcagatgtcatccagcgaggatgacaacacgaacaccatcatcaacaatagcaacaataaggataacaacaataatcagtacacaatcaataaaattataccagaacaacacagcagacagcggcaacatgtacagcagcaacatctgcagcagcagcagcagcagcatctgcaacagcagcaacatcagcggcagcagcaacagcagcaacgtgaggaggagagcgagtcggaaagggaagcggaaccggatcgagaacaacaattgttgcatcaaaagccaaaggatgtcattacgataaagcagctgccattagctgggagcaacaataacaacatcaacaataacatcaacaacaacaacaacaacaacaacatcatggaggaccccatggcggatacatga

SEQ ID NO: 25 - protein, 500 aa, Arabidopsis thaliana
MQMNSVWKLSLGLLLLSSVIGSFAELDFGHCETLVKKWADSSSSREEHVNKDKRSLKDLLFFLHVPRTGGRTYFHCFLRKLYDSSEECPRSYDKLHFNPRKEKCKLLATHDDYSLMAKLPRERTSVMTIVRDPIARVLSTYEFSVEVAARFLVHPNLTSASRMSSRIRKSNVISTLDIWPWKYLVPWMREDLFARRDARKLKEVVIIEDDNPYDMEEMLMPLHKYLDAPTAHDIIHNGATFQIAGLTNNSHLSEAHEVRHCVQKFKSLGESVLQVAKRRLDSMLYVGLTEEHRESASLFANVVGSQVLSQVVPSNATAKIKALKSEASVTISETGSDKSNIQNGTSEVTLNKAEAKSGNMTVKTLMEVYEGCITHLRKSQGTRRVNSLKRITPANFTRGTRTRVPKEVIQQIKSLNNLDVELYKYAKVIFAKEHELVSNKLISSSKRSIVDLPSELKSVLGEMGEEKLWKFVPVALMLLLIVLFFLFVNAKRRRTSKVKI

SEQ ID NO: 26 - DNA, 1503 bp, Arabidopsis thaliana
atgcaaatgaactctgtttggaagctgtctcttgggttattacttcttagctcagttattggctcttttgcggaacttgattttggccattgcgaaactcttgtgaaaaaatgggctgattcttcttcatctcgtgaagaacatgttaataaagacaaacgctcgcttaaggatttgctcttctttctccacgttccgcgaactggaggcagaacatattttcattgttttttgaggaagttgtatgatagctctgaggaatgtcctcgatcttacgacaagctccacttcaatccaaggaaggaaaagtgcaagttgttagccacacatgatgattatagtttgatggcaaagcttccgagggagagaacttcggtgatgacaatagttcgggatcctattgcgcgtgtgttaagcacttatgaattttccgtagaggtagcagctaggtttttggtgcatcccaatttaacttctgcgtcaaggatgtctagccgcatacgcaagagtaatgtaataagcacactagacatatggccatggaaatacctagttccatggatgagagaagacttgtttgctcggcgagatgcacgaaaattgaaggaggtagtgatcattgaggacgataacccgtatgacatggaggagatgcttatgcctttgcacaaatatcttgatgcgcctactgctcatgacatcatccacaatggagcgacttttcagattgcaggattgacaaataactcccatttatcagaagcacacgaggttcggcattgtgtgcagaaattcaaaagccttggtgagtctgttctccaagttgccaagaggaggctagacagcatgttgtatgttggactgacagaggagcacagggaatctgcatcactttttgccaatgtagtgggttctcaagtgctgtctcaagtggttccgtccaatgcaactgcgaaaatcaaagctcttaaatcagaagcaagtgtcacaatttcagaaaccgggtcagataagagtaatattcagaatggtacatctgaagttacattgaataaggcagaagctaagagtgggaatatgacggtaaaaacccttatggaagtctatgaaggctgcatcactcatttacgaaagtcccaaggaaccagacgggtcaactctctgaagagaataactccagcaaattttacaagagggacgcgtacaagagttcctaaagaggtcattcagcagatcaaatcgcttaacaacctcgatgtggagctctacaaatatgcaaaagtaatctttgccaaagaacatgaattagtgtcgaataagttgatctcaagttctaagagaagcattgttgatctgccgagtgagttaaagagcgtattgggagaaatgggtgaagagaagctatggaagttcgtaccagtggcattgatgcttttattgatcgtcctcttctttctatttgtaaacgctaaaaggagaagaacctccaaagttaagatttga

SEQ ID NO: 27 - protein, 23 aa, Artificial Sequence
  PG9 fragment
NGYNYYDFYDGYYNYHYMDVWGK

SEQ ID NO: 28 - protein, 6 aa, Artificial Sequence
  PG9 fragment
NGYNYY

SEQ ID NO: 29 - protein, 12 aa, Artificial Sequence
  PG9 fragment
DFYDGYYNYHYM

SEQ ID NO: 30 - protein, 240 aa, Artificial Sequence
  PG9LC
MANKHLSLSLFLVLLGLSASLASGQSALTQPASVSGSPGQSITISCQGTSNDVGGYESVSWYQQHPGKAPKVVIYDVSKRPSGVSNRFSGSKSGNTASLTISGLQAEDEGDYYCKSLTSTRRRVFGTGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 31 - DNA, 723 bp, Artificial Sequence
  PG9LC
atggcgaacaaacacttgtccctctccctcttcctcgtcctccttggcctgtcggccagcttggcctcaggtcagagtgctcttactcagcctgcttctgtttctggttctcctggtcagagcatcaccatttcttgccagggaacctctaacgatgtgggaggttacgagtccgtgtcttggtatcaacagcatcctggtaaggctcctaaggtggtgatctacgatgtgagcaagaggccttctggtgtgagcaataggttcagcggtagcaagtctggtaacaccgcttctcttaccatctctggacttcaggctgaggatgagggagattactactgcaagtctctgacctccactagaagaagggtgttcggaaccggtactaagcttactgttctgggtcaacctaaggctgctccttctgtgactttgttccctccatcttctgaggaactgcaggctaacaaggctacccttgtgtgcctgatcagcgatttttaccctggtgctgttaccgtggcttggaaggctgattcttcacctgttaaggctggtgtggaaaccaccactcctagcaagcagagcaacaacaagtacgctgctagctcctaccttagccttactcctgaacagtggaagtcccacaagagctactcatgccaggttacccatgagggttctaccgtggaaaagactgttgctcctactgagtgcagctag

SEQ ID NO: 32 - protein, 240 aa, Artificial Sequence
  PG9LC-RSH
MANKHLSLSLFLVLLGLSASLASGQSALTQPASVSGSPGQSITISCQGTSNDVGGYESVSWYQQHPGKAPKVVIYDVSKRPSGVSNRFSGSKSGNTASLTISGLQAEDEGDYYCKSLTSRSHRVFGTGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 33 - DNA, 723 bp, Artificial Sequence
  PG9LC-RSH
atggcgaacaaacacttgtccctctccctcttcctcgtcctccttggcctgtcggccagcttggcctcaggtcagagtgctcttactcagcctgcttctgtttctggttctcctggtcagagcatcaccatttcttgccagggaacctctaacgatgtgggaggttacgagtccgtgtcttggtatcaacagcatcctggtaaggctcctaaggtggtgatctacgatgtgagcaagaggccttctggtgtgagcaataggttcagcggtagcaagtctggtaacaccgcttctcttaccatctctggacttcaggctgaggatgagggagattactactgcaagtctctgacctccagaagtcacagggtgttcggaaccggtactaagcttactgttctgggtcaacctaaggctgctccttctgtgactttgttccctccatcttctgaggaactgcaggctaacaaggctacccttgtgtgcctgatcagcgatttttaccctggtgctgttaccgtggcttggaaggctgattcttcacctgttaaggctggtgtggaaaccaccactcctagcaagcagagcaacaacaagtacgctgctagctcctaccttagccttactcctgaacagtggaagtcccacaagagctactcatgccaggttacccatgagggttctaccgtggaaaagactgttgctcctactgagtgcagctag

SEQ ID NO: 34 - DNA, 1392 bp, Artificial Sequence
  gp120ZM109
atgcctatgggcagcctgcagcccctggccacactgtatctgctgggaatgctggtggccagctgcctgggcgtgtggaaagaggccaagaccaccctgttctgcgccagcgacgccaagagctacgagcgcgaggtgcacaatgtgtgggccacccatgcctgcgtgcccaccgatcctgatccccaggaactcgtgatggccaacgtgaccgagaacttcaacatgtggaagaacgacatggtggaccagatgcacgaggacatcatcagcctgtgggaccagagcctgaagccctgcgtgaagctgacccctctgtgcgtgaccctgaactgcacatctcctgccgcccacaacgagagcgagacaagagtgaagcactgcagcttcaacatcaccaccgacgtgaaggaccggaagcagaaagtgaacgccaccttctacgacctggacatcgtgcccctgagcagcagcgacaacagcagcaacagctccctgtacagactgatcagctgcaacaccagcaccatcacccaggcctgccccaaggtgtccttcgaccccatccccatccactactgtgcccctgccggctacgccatcctgaagtgcaacaacaagaccttcagcggcaagggcccctgcagcaacgtgtccaccgtgcagtgtacccacggcatcagacccgtggtgtccacccagctgctgctgaatggcagcctggccgaagaggaaatcgtgatcagaagcgagaacctgaccgacaacgccaagacaatcattgtgcatctgaacaagagcgtggaaatcgagtgcatcaggcccggcaacaacaccagaaagagcatcagactgggccctggccagaccttttacgccaccggggatgtgatcggcgacatccggaaggcctactgcaagatcaacggcagcgagtggaacgagacactgacaaaggtgtccgagaagctgaaagagtactttaacaagaccattcgcttcgcccagcactctggcggcgacctggaagtgaccacccacagcttcaattgcagaggcgagttcttctactgcaataccagcgagctgttcaacagcaacgccaccgagagcaatatcaccctgccctgccggatcaagcagatcatcaatatgtggcagggcgtgggcagagctatgtacgcccctcccatccggggcgagatcaagtgcacctctaacatcaccggcctgctgctgaccagggacggcggaaacaacaacaatagcaccgaggaaatcttccggcccgagggcggcaacatgagagacaattggagatccgagctgtacaagtacaaggtggtggaaatcaagggcctgcggggcagccaccaccatcatcaccattga

SEQ ID NO: 35 - protein, 463 aa, Artificial Sequence
  gp120ZM109
MPMGSLQPLATLYLLGMLVASCLGVWKEAKTTLFCASDAKSYEREVHNVWATHACVPTDPDPQELVMANVTENFNMWKNDMVDQMHEDIISLWDQSLKPCVKLTPLCVTLNCTSPAAHNESETRVKHCSFNITTDVKDRKQKVNATFYDLDIVPLSSSDNSSNSSLYRLISCNTSTITQACPKVSFDPIPIHYCAPAGYAILKCNNKTFSGKGPCSNVSTVQCTHGIRPVVSTQLLLNGSLAEEEIVIRSENLTDNAKTIIVHLNKSVEIECIRPGNNTRKSIRLGPGQTFYATGDVIGDIRKAYCKINGSEWNETLTKVSEKLKEYFNKTIRFAQHSGGDLEVTTHSFNCRGEFFYCNTSELFNSNATESNITLPCRIKQIINMWQGVGRAMYAPPIRGEIKCTSNITGLLLTRDGGNNNNSTEEIFRPEGGNMRDNWRSELYKYKVVEIKGLRGSHHHHHH